(19) United States
(12) Patent Application Publication
(10) Pub. No.: US 2009/0088419 A1
(43) Pub. Date: Apr. 2, 2009

(54) PYRIDYL ACETIC ACID COMPOUNDS

(75) Inventors: Hironobu Maezaki, Osaka-shi (JP); Nobuhiro Suzuki, Tsukuba-shi (JP)

Correspondence Address: EDWARDS ANGELL PALMER & DODGE LLP, P.O. BOX 55874, BOSTON, MA, 02205, US

(73) Assignee: TAKEDA PHARMACEUTICAL COMPANY LIMITED, Osaka-shi, Osaka (JP)

(21) Appl. No.: 11/817,088

(22) PCT Filed: Feb. 24, 2006
(86) PCT No.: PCT/JP2006/304177

§ 371 Date: Aug. 24, 2007

(30) Foreign Application Priority Data
Feb. 25, 2005 (JP) 2005-052018

(51) Int. Cl. C07D 213/02; A61K 31/4439; C07D 401/02; A61K 31/445; A61K 31/496; A61P 3/10; A61K 31/4545; A61K 31/551; A61K 31/55

(52) U.S. Cl. 514/217.04; 546/329; 514/357; 514/343; 546/276.4; 540/597; 546/277.1; 514/339; 514/318; 546/194; 514/311; 514/218; 540/575; 544/361; 514/253.01; 514/278

(57) ABSTRACT

The present invention provides a compound represented by the formula (I): wherein R₁ is a C?1-6#191 alkyl group optionally substituted by a C?3-10#191 cycloalkyl group, R₂ is a C?2-6#191 alkyl group, R₃ is a hydrogen atom, a C?1-6#191 alkyl group or a halogen atom, and X is —OR₆ or —NR₄R₅ wherein R₄ and R₆ are each independently a hydrogen atom, an optionally substituted hydrocarbon group or an optionally substituted heterocyclic group, R₅ is an optionally substituted hydrocarbon group, an optionally substituted heterocyclic group or an optionally substituted hydroxy group, or R₄ and R₅ optionally form, together with the adjacent nitrogen atom, an optionally substituted nitrogen-containing heterocycle, or a salt thereof. The compound of the present invention has a superior peptidase inhibitory action and is useful as an agent for the prophylaxis or treatment of diabetes and the like.

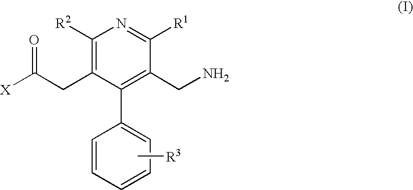

### TECHNICAL FIELD

The present invention relates to a pyridyl acetic acid compound having a peptidase inhibitory activity, which is useful as an agent for the prophylaxis or treatment of diabetes and the like.

### BACKGROUND ART

Peptidase is known to relate to various diseases. Dipeptidyl dipeptidase-IV (hereinafter sometimes to be abbreviated as DPP-IV), which is one kind of peptidases, is serine protease that specifically binds with a peptide containing proline (or alanine) at the 2nd from the N-terminal and cleaves the C-terminal side of the proline (or alanine) to produce dipeptide. DPP-IV has been shown to be the same molecule as CD26, and reported to be also involved in the immune system. While the role of DPP-IV in mammals has not been entirely clarified, it is considered to play an important role in the metabolism of neuropeptides, activation of T cells, adhesion of cancer cells to endothelial cells, invasion of HIV into cells and the like. Particularly, from the aspect of glycometabolism, DPP-IV is involved in the inactivation of GLP-1 (glucagon-like peptide-1) and GIP (Gastric inhibitory peptide/Glucose-dependent insulinotropic peptide), which are incretins. With regard to GLP-1, moreover, it is known that the physiological activity of GLP-1 is markedly impaired because it has a short plasma half-life of 1-2 minutes, and GLP-1 (9-36)amide, which is a degradation product by DPP-IV, acts on GLP-1 receptor as an antagonist, thus decomposing GLP-1 by DPP-IV. It is also known that suppression of degradation of GLP-1 by inhibiting DPP-IV activity leads to potentiation of physiological activity that GLP-1 shows, such as glucose concentration-dependent insulin secretagogue effect and the like. From these facts, a compound having a DPP-IV inhibitory activity is expected to show effect on impaired glucose tolerance, postprandial hyperglycemia and fasting hyperglycemia observed in type I and type II diabetes and the like, obesity or diabetic complications associated therewith and the like.

As a compound having a DPP-IV inhibitory action, for example, a compound represented by the formula

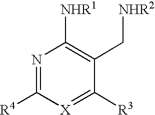

wherein X is N or CR⁵ (wherein R⁵ is hydrogen or lower alkyl); R¹ and R² are independently hydrogen or lower alkyl; R³ is heterocyclic group or aryl, each optionally substituted by lower alkyl and the like; R⁴ is lower alkyl and the like, or a salt thereof, has been reported (see WO03/068757).

However, there is no report on the compound of the present invention.

### DISCLOSURE OF THE INVENTION

There is a demand for the development of a compound having a peptidase inhibitory action, which is useful as an agent for the prophylaxis or treatment of diabetes and the like and superior in efficacy, duration of action, specificity, lower toxicity and the like.

The present inventors have first found that a compound represented by the formula (I):

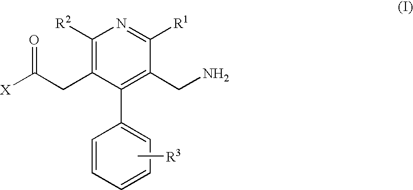

wherein
R¹ is a C₁₋₆ alkyl group optionally substituted by a C₃₋₁₀ cycloalkyl group,
R² is a C₂₋₆ alkyl group,
R³ is a hydrogen atom, a C₁₋₆ alkyl group or a halogen atom, and
X is —OR⁶ or —NR⁴R⁵
   wherein R⁴ and R⁶ are each independently a hydrogen atom, an optionally substituted hydrocarbon group or an optionally substituted heterocyclic group, R⁵ is an optionally substituted hydrocarbon group, an optionally substituted heterocyclic group or an optionally substituted hydroxy group, or R⁴ and R⁵ optionally form, together with the adjacent nitrogen atom, an optionally substituted nitrogen-containing heterocycle, or a salt thereof
      [hereinafter sometimes to be abbreviated as compound (I)], which is characterized by a chemical structure wherein an amino group is bonded to the 3-position via a methylene group; an optionally substituted phenyl group is bonded to the 4-position; an acyl group is bonded to the 5-position via a methylene group; and a C₂₋₆ alkyl group is bonded to the 6-position, of the pyridine ring, has a superior peptidase inhibitory action and is useful as an agent for the prophylaxis or treatment of diabetes and the like. Based on this finding, the present inventors have conducted intensive studies and completed the present invention.

Accordingly, the present invention relates to
1) compound (I);
2) compound (I), wherein X is —OH;
3) compound (I), wherein R¹ is a C₃₋₆ alkyl group;
4) compound (I), wherein R³ is a C₁₋₆ alkyl group;
5) compound (I), which is
[5-(aminomethyl)-2-ethyl-6-isobutyl-4-(4-methylphenyl)pyridin-3-yl]acetic acid;
[5-(aminomethyl)-2,6-diisobutyl-4-(4-methylphenyl)pyridin-3-yl]acetic acid;
[5-(aminomethyl)-2-ethyl-4-(4-methylphenyl)-6-neopentylpyridin-3-yl]acetic acid; or
1-{[5-(aminomethyl)-2-ethyl-4-(4-methylphenyl)-6-neopentylpyridin-3-yl]acetyl}-L-prolinamide; or a salt thereof;
6) a prodrug of compound (I);
7) a pharmaceutical agent comprising compound (I) or a prodrug thereof;
8) the pharmaceutical agent of the aforementioned 7), which is an agent for the prophylaxis or treatment of diabetes, diabetic complications, impaired glucose tolerance or obesity;
9) a peptidase inhibitor comprising compound (I) or a prodrug thereof;
10) the inhibitor of the aforementioned 9), wherein the peptidase is dipeptidyl peptidase-IV;
11) use of compound (I) or a prodrug thereof for the production of an agent for the prophylaxis or treatment of diabetes, diabetic complications, impaired glucose tolerance or obesity;
12) use of compound (I) or a prodrug thereof for the production of a peptidase inhibitor;
13) a method of preventing or treating diabetes, diabetic complications, impaired glucose tolerance or obesity in a mammal, which comprises administering compound (I) or a prodrug thereof to said mammal;
14) a method of inhibiting peptidase in a mammal, which comprises administering compound (I) or a prodrug thereof to said mammal;
15) a method of producing a compound represented by the formula (I-a):

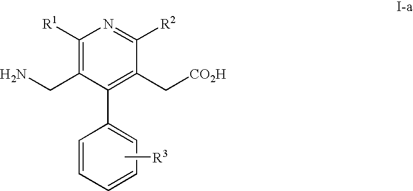

wherein
R¹ is a C₁₋₆ alkyl group optionally substituted by a C₃₋₁₀ cycloalkyl group,
R² is a C₂₋₆ alkyl group, and
R³ is a hydrogen atom, a C₁₋₆ alkyl group or a halogen atom, or a salt thereof, which comprises subjecting a compound represented by the formula (1):

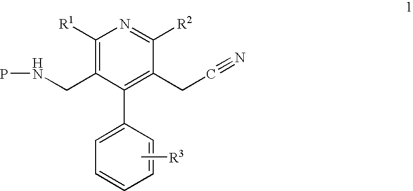

wherein
P is a hydrogen atom or an amino-protecting group, and
R¹, R² and R³ are each as defined above, or a salt thereof, to hydrolysis and deprotection;
and the like.

The compound of the present invention has a superior peptidase inhibitory action and is useful as an agent for the prophylaxis or treatment of diabetes and the like.

### BEST MODE FOR EMBODYING THE INVENTION

Each symbol in the formula (I) is described in detail in the following.

As the “C₁₋₆ alkyl group” of the “C₁₋₆ alkyl group optionally substituted by a C₃₋₁₀ cycloalkyl group” for R¹, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like can be mentioned.

As the “C₃₋₁₀ cycloalkyl group” of the “C₁₋₆ alkyl group optionally substituted by a C₃₋₁₀ cycloalkyl group” for R¹, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

R¹ is preferably a C₃₋₆ alkyl group, more preferably a branched C₃₋₆ alkyl group, particularly preferably isobutyl or neopentyl.

As the “C₂₋₆ alkyl group” for R², for example, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like can be mentioned.

R² is preferably ethyl or isobutyl.

As the “C₁₋₆ alkyl group” for R³, those exemplified for the aforementioned R¹ can be mentioned.

As the “halogen atom” for R³, for example, fluorine, chlorine, bromine and iodine can be mentioned.

R³ is preferably a C₁₋₆ alkyl group, more preferably methyl.

As the “hydrocarbon group” of the “optionally substituted hydrocarbon group” for R⁴, R⁵ or R⁶, for example, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₃ aralkyl group, a C₈₋₁₃ arylalkenyl group, a C₃₋₁₀ cycloalkyl-C₁₋₆ alkyl group and the like can be mentioned.

Here, as the C₁₋₁₀ alkyl group, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

As the C₂₋₁₀ alkenyl group, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

As the C₂₋₁₀ alkynyl group, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

As the C₃₋₁₀ cycloalkyl group, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned.

The above-mentioned C₃₋₁₀ cycloalkyl group, C₃₋₁₀ cycloalkenyl group and C₄₋₁₀ cycloalkadienyl group are each optionally condensed with a benzene ring, and, for example, indanyl, dihydronaphthyl, tetrahydronaphthyl, fluorenyl and the like can be mentioned.

As the C₆₋₁₄ aryl group, for example, phenyl, naphthyl, anthryl, phenanthryl, acenaphthylenyl, biphenylyl and the like can be mentioned. Of these, phenyl, 1-naphthyl, 2-naphthyl and the like are preferable.

As the C₇₋₁₃ aralkyl group, for example, benzyl, phenethyl, naphthylmethyl, biphenylylmethyl and the like can be mentioned.

As the C₈₋₁₃ arylalkenyl group, for example, styryl and the like can be mentioned.

As the C₃₋₁₀ cycloalkyl-C₁₋₆ alkyl group, for example, cyclohexylmethyl and the like can be mentioned.

The aforementioned C₁₋₁₀ alkyl group, C₂₋₁₀ alkenyl group and C₂₋₁₀ alkynyl group optionally have 1 to 3 substituents at substitutable positions.

As such substituents, for example,
(1) a C₃₋₁₀ cycloalkyl group (e.g., cyclopropyl, cyclohexyl);
(2) a C₆₋₁₄ aryl group (e.g., phenyl, naphthyl);
(3) an aromatic heterocyclic group (e.g., thienyl, furyl, pyridyl, oxazolyl, thiazolyl, tetrazolyl, oxadiazolyl, pyrazinyl, quinolyl, indolyl) optionally substituted by 1 to 3 substituents selected from a carboxyl group, a carbamoyl group, a thiocarbamoyl group and a C₁₋₆ alkoxy-carbonyl group (e.g., methoxycarbonyl, ethoxycarbonyl, propoxycarbonyl, tert-butoxycarbonyl);
(4) a non-aromatic heterocyclic group (e.g., tetrahydrofuryl, morpholino, thiomorpholino, piperidino, pyrrolidinyl, piperazinyl, oxodioxolyl, oxodioxolanyl, oxo-2-benzofuranyl, oxooxadiazolyl) optionally substituted by a C₁₋₆ alkyl group (e.g., methyl, ethyl);
(5) an amino group optionally mono- or di-substituted by substituent(s) selected from a C₁₋₆ alkyl group (e.g., methyl, ethyl), a C₁₋₆ alkyl-carbonyl group (e.g., acetyl, isobutanoyl, isopentanoyl) and a C₁₋₆ alkoxy-carbonyl group (e.g., methoxycarbonyl, ethoxycarbonyl, propoxycarbonyl, tert-butoxycarbonyl);
(6) a C₁₋₆ alkylsulfonylamino group (e.g., methylsulfonylamino);
(7) an amidino group;
(8) a C₁₋₆ alkyl-carbonyl group (e.g., acetyl, isobutanoyl, isopentanoyl);
(9) a C₁₋₆ alkoxy-carbonyl group (e.g., methoxycarbonyl, ethoxycarbonyl, propoxycarbonyl, tert-butoxycarbonyl);
(10) a C₁₋₆ alkylsulfonyl group (e.g., methylsulfonyl, ethylsulfonyl);
(11) a carbamoyl group optionally mono- or di-substituted by C₁₋₆ alkyl group(s) (e.g., methyl, ethyl) optionally substituted by 1 to 3 halogen atoms (e.g., fluorine, chlorine, bromine, iodine);
(12) a thiocarbamoyl group optionally mono- or di-substituted by C₁₋₆ alkyl group(s) (e.g., methyl, ethyl) optionally substituted by 1 to 3 halogen atoms (e.g., fluorine, chlorine, bromine, iodine);
(13) a sulfamoyl group optionally mono- or di-substituted by C₁₋₆ alkyl group(s) (e.g., methyl, ethyl) optionally substituted by 1 to 3 halogen atoms (e.g., fluorine, chlorine, bromine, iodine);
(14) a carboxyl group;
(15) a hydroxy group;
(16) a C₁₋₆ alkoxy group (e.g., methoxy, ethoxy) optionally substituted by 1 to 3 halogen atoms (e.g., fluorine, chlorine, bromine, iodine);
(17) a C₂₋₆ alkenyloxy group (e.g., ethenyloxy) optionally substituted by 1 to 3 halogen atoms (e.g., fluorine, chlorine, bromine, iodine);
(18) a C₃₋₁₀ cycloalkyloxy group (e.g., cyclohexyloxy);
(19) a C₇₋₁₃ aralkyloxy group (e.g., benzyloxy);
(20) a C₆₋₁₄ aryloxy group (e.g., phenyloxy, naphthyloxy);
(21) a C₁₋₆ alkyl-carbonyloxy group (e.g., acetyloxy, tert-butylcarbonyloxy);
(22) a thiol group;
(23) a C₁₋₆ alkylthio group (e.g., methylthio, ethylthio) optionally substituted by 1 to 3 halogen atoms (e.g., fluorine, chlorine, bromine, iodine);
(24) a C₇₋₁₃ aralkylthio group (e.g., benzylthio);
(25) a C₆₋₁₄ arylthio group (e.g., phenylthio, naphthylthio);
(26) a sulfo group;
(27) a cyano group;
(28) an azido group;
(29) a nitro group;
(30) a nitroso group;
(31) a halogen atom (e.g., fluorine, chlorine, bromine, iodine);
(32) a C₁₋₆ alkylsulfinyl group (e.g., methylsulfinyl); and the like can be mentioned.

The C₃₋₁₀ cycloalkyl group, C₃₋₁₀ cycloalkenyl group, C₄₋₁₀ cycloalkadienyl group, C₆₋₁₄ aryl group, C₇₋₁₃ aralkyl group, C₈₋₁₃ arylalkenyl group and C₃₋₁₀ cycloalkyl-C₁₋₆ alkyl group, which are exemplarily recited for the aforementioned “hydrocarbon group”, optionally have 1 to 3 substituents at substitutable positions.

As such substituents, for example, those exemplarily recited for the substituents for the aforementioned C₁₋₁₀ alkyl group and the like; a C₁₋₆ alkyl group (e.g., methyl, ethyl) optionally substituted by 1 to 3 substituents selected from a halogen atom (e.g., fluorine, chlorine, bromine, iodine), a carboxyl group, a C₁₋₆ alkoxy-carbonyl group (e.g., methoxycarbonyl, ethoxycarbonyl), a carbamoyl group and a C₁₋₆ alkoxy group (e.g., methoxy); a C₂₋₆ alkenyl group (e.g., ethenyl, 1-propenyl) optionally substituted by 1 to 3 substituents selected from a halogen atom (e.g., fluorine, chlorine, bromine, iodine), a carboxyl group, a C₁₋₆ alkoxy-carbonyl group (e.g., methoxycarbonyl, ethoxycarbonyl) and a carbamoyl group;

a C₇₋₁₃ aralkyl group (e.g., benzyl);
an oxo group;
and the like can be mentioned.

As the “heterocyclic group” of the “optionally substituted heterocyclic group” for R⁴, R⁵ or R⁶, an aromatic heterocyclic group and a non-aromatic heterocyclic group can be mentioned.

As the aromatic heterocyclic group, for example, a 5- to 7-membered monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group can be mentioned. As the fused aromatic heterocyclic group, for example, a group wherein these 5- to 7-membered monocyclic aromatic heterocyclic groups and a 6-membered ring containing 1 or 2 nitrogen atoms, a benzene ring or a 5-membered ring containing one sulfur atom are fused, and the like can be mentioned.

As preferable examples of the aromatic heterocyclic group, monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl, oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl) and the like;

fused aromatic heterocyclic groups such as quinolyl (e.g., 2-quinolyl, 3-quinolyl, 4-quinolyl), quinazolyl (e.g., 2-quinazolyl, 4-quinazolyl), quinoxalyl (e.g., 2-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzothiazolyl (e.g., 2-benzothiazolyl), benzothiadiazolyl (e.g., benzo[1,2,5]thiadiazol-4-yl), benzimidazolyl (e.g., benzimidazol-1-yl, benzimidazol-2-yl), indolyl (e.g., indol-1-yl, indol-3-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl), imidazopyrazinyl (e.g., 1H-imidazo[4,5-b]pyrazin-2-yl) and the like,
and the like can be mentioned.

As the non-aromatic heterocyclic group, for example, a 5- to 7-membered monocyclic non-aromatic heterocyclic group containing, as a ring-constituting atom besides carbon atoms, 1 to 4 heteroatoms selected from an oxygen atom, a sulfur atom and a nitrogen atom, and a fused non-aromatic heterocyclic group can be mentioned. As the fused non-aromatic heterocyclic group, for example, a group wherein these 5- to 7-membered monocyclic non-aromatic heterocyclic groups and a 6-membered ring containing 1 or 2 nitrogen atoms, a benzene ring or a 5-membered ring containing one sulfur atom are fused, and the like can be mentioned.

As preferable examples of the non-aromatic heterocyclic group, pyrrolidinyl (e.g., 1-pyrrolidinyl, 2-pyrrolidinyl, 3-pyrrolidinyl), piperidinyl (e.g., piperidino), morpholinyl (e.g., morpholino), thiomorpholinyl (e.g., thiomorpholino), piperazinyl (e.g., 1-piperazinyl), hexamethyleniminyl (e.g., hexamethylenimin-1-yl), oxazolidinyl (e.g., oxazolidin-3-yl), thiazolidinyl (e.g., thiazolidin-3-yl), imidazolidinyl (e.g., imidazolidin-3-yl), oxoimidazolidinyl (e.g., 2-oxoimidazolidin-1-yl), dioxoimidazolidinyl (e.g., 2,4-dioxoimidazolidin-3-yl), dioxooxazolidinyl (e.g., 2,4-dioxooxazolidin-3-yl, 2,4-dioxooxazolidin-5-yl, 2,4-dioxooxazolidin-1-yl), dioxothiazolidinyl (e.g., 2,4-dioxothiazolidin-3-yl, 2,4-dioxothiazolidin-5-yl), dioxoisoindolinyl (e.g., 1,3-dioxoisoindolin-2-yl), oxooxadiazolidinyl (e.g., 5-oxooxadiazolidin-3-yl), oxothiadiazolidinyl (e.g., 5-oxothiadiazolidin-3-yl), oxopiperazinyl (e.g., 3-oxopiperazin-1-yl), dioxopiperazinyl (e.g., 2,3-dioxopiperazin-1-yl, 2,5-dioxopiperazin-1-yl), oxodioxolyl (e.g., 2-oxo-1,3-dioxol-4-yl), oxodioxolanyl (e.g., 2-oxo-1,3-dioxolan-4-yl), 3-oxo-1,3-dihydro-2-benzofuranyl (e.g., 3-oxo-1,3-dihydro-2-benzofuran-1-yl), oxodihydrooxadiazolyl (e.g., 5-oxo-4,5-dihydro-1,2,4-oxadiazol-3-yl), oxodihydropyrazolyl (e.g., 5-oxo-4,5-dihydro-1H-pyrazol-3-yl), 4-oxo-2-thioxo-1,3-thiazolidin-5-yl, 4-oxo-2-thioxo-1,3-oxazolidin-5-yl, tetrahydropyranyl (e.g., 4-tetrahydropyranyl), 4-oxo-4,5,6,7-tetrahydro-1-benzofuranyl (e.g., 4-oxo-4,5,6,7-tetrahydro-1-benzofuran-3-yl), 1,3(2H, 5H)-dioxotetrahydroimidazo[1,5-a]pyridinyl, 1,3(2H, 5H)-dioxo-10,10a-dihydroimidazo[1,5-b]isoquinolinyl, azabicyclooctyl (e.g., 1-azabicyclo[2.2.2]octan-2-yl, 1-azabicyclo[2.2.2]octan-3-yl) and the like can be mentioned.

As the “nitrogen-containing heterocycle” of the “optionally substituted nitrogen-containing heterocycle” formed by R⁴ and R⁵ together with the adjacent nitrogen atom, for example, a 5- to 7-membered nitrogen-containing heterocycle containing, as a ring-constituting atom besides carbon atoms, at least one nitrogen atom and optionally further containing 1 or 2 heteroatoms selected from an oxygen atom, a sulfur atom and a nitrogen atom can be mentioned. As preferable examples of the “nitrogen-containing heterocycle”, pyrrolidine, imidazolidine, pyrazolidine, piperidine, piperazine, morpholine, thiomorpholine, oxopiperazine, homopiperidine, homopiperazine, thiazolidine, dihydroindole (e.g., 2,3-dihydroindole), dihydroisoindole (e.g., 1,3-dihydroisoindole), tetrahydroquinoline (e.g., 1,2,3,4-tetrahydroquinoline), triazaspirodecanedione (e.g., 1,3,8-triazaspiro[4.5]decane-2,4-dione), hexahydropyrazinooxazinone (e.g., hexahydropyrazino[2,1-c][1,4]oxazin-4(3H)-one) and the like can be mentioned.

The nitrogen-containing heterocycle optionally has 1 to 3 (preferably 1 or 2) substituents at substitutable positions. As such substituents, for example, those exemplarily recited for the substituents for the C₃₋₁₀ cycloalkyl group, which is exemplarily recited for the aforementioned “hydrocarbon group” of the “optionally substituted hydrocarbon group” for R⁴ or R⁵, can be mentioned.

As the “optionally substituted hydroxy group” for R⁵, for example, a hydroxy group optionally substituted by a hydrocarbon group can be mentioned.

As the hydrocarbon group, here, those exemplarily recited for the aforementioned “hydrocarbon group” of the “optionally substituted hydrocarbon group” for R⁴, R⁵ or R⁶ can be mentioned.

The “optionally substituted hydroxy group” is preferably a hydroxy group, a C₁₋₆ alkoxy group (e.g., methoxy, ethoxy) and the like.

R⁴ and R⁵ are preferably the same or different and each is a hydrogen atom (only for R⁴), an optionally substituted C₁₋₁₀ alkyl group, an optionally substituted C₃₋₁₀ cycloalkyl group, an optionally substituted C₆₋₁₄ aryl group, an optionally substituted C₇₋₁₃ aralkyl group, an optionally substituted heterocyclic group or an optionally substituted hydroxy group (only for R⁵). To be specific,

(1) a hydrogen atom (only for R⁴);
(2) a C₁₋₁₀ alkyl group (preferably methyl, ethyl) optionally substituted by 1 to 3 substituents selected from a C₁₋₆ alkoxy-carbonyl group, a C₁₋₆ alkoxy group and a heterocyclic group (e.g., 2-thienyl);
(3) a C₃₋₁₀ cycloalkyl group optionally condensed with a benzene ring (preferably indanyl, tetrahydronaphthyl);
(4) a C₆₋₁₄ aryl group (preferably phenyl) optionally substituted by 1 to 3 substituents selected from

(4a) a halogen atom;

(4b) a C₁₋₆ alkyl group optionally substituted by a C₁₋₆ alkoxy-carbonyl group;

(4c) a C₁₋₆ alkyl-carbonyl group;

(4d) a C₁₋₆ alkylsulfonyl group; and

(4e) a C₁₋₆ alkoxy group optionally substituted by 1 to 3 halogen atoms (e.g., fluorine, chlorine, bromine, iodine);

(5) a C₇₋₁₃ aralkyl group (preferably benzyl) optionally substituted by 1 to 3 C₁₋₆ alkylsulfonyl groups;
(6) a heterocyclic group (e.g., pyridyl, isoxazolyl, pyrazolyl, thiadiazolyl, benzothiadiazolyl, oxodihydropyrazolyl, azabicyclooctyl, pyrrolidinyl) optionally substituted by 1 to 3 substituents selected from a C₁₋₆ alkyl group, a C₆₋₁₄ aryl group, a C₇₋₁₃ aralkyl group and a C₁₋₆ alkoxy-carbonyl group; and
(7) a C₁₋₆ alkoxy group (only for R⁵);
are preferable.

As the “nitrogen-containing heterocycle” of the “optionally substituted nitrogen-containing heterocycle” formed by R⁴ and R⁵ together with the adjacent nitrogen atom, for example, pyrrolidine, piperidine, piperazine, morpholine, homopiperidine, homopiperazine, thiazolidine, dihydroindole, dihydroisoindole, tetrahydroquinoline, triazaspirodecanedione, hexahydropyrazinooxazinone and the like are preferable.

As the substituents for the nitrogen-containing heterocycle,

(1) a carbamoyl group;
(2) a C₁₋₆ alkyl-carbonyl group;
(3) a C₁₋₆ alkoxy-carbonyl group;
(4) a C₁₋₆ alkylsulfonyl group;
(5) a C₁₋₆ alkyl group optionally substituted by 1 to 3 substituents selected from a C₁₋₆ alkoxy group and a C₁₋₆ alkoxy-carbonyl group;
(6) a non-aromatic heterocyclic group (e.g., pyrrolidinyl);
(7) an amino group optionally mono- or di-substituted by C₁₋₆ alkyl-carbonyl group(s); and
(8) a halogen atom (e.g., fluorine, chlorine, bromine, iodine); are preferable.

R⁶ is preferably a hydrogen atom or an optionally substituted C₁₋₁₀ alkyl group. As the substituents for the C₁₋₁₀ alkyl group, a non-aromatic heterocyclic group (e.g., oxodioxolyl) optionally substituted by a C₁₋₆ alkyl group can be mentioned. R⁶ is particularly preferably a hydrogen atom.

As preferable examples of compound (I), the following compounds can be mentioned.

### [Compound A]

A compound wherein

R¹ is a C₃₋₆ alkyl group (preferably isobutyl, neopentyl);
R² is a C₂₋₆ alkyl group (preferably ethyl, isobutyl);
R³ is a C₁₋₆ alkyl group (preferably methyl);

### X is —OR⁶ or —NR⁴R⁵,

R⁶ is a hydrogen atom, or a C₁₋₁₀ alkyl group optionally substituted by a non-aromatic heterocyclic group (e.g., oxodioxolyl) optionally substituted by a C₁₋₆ alkyl group;
R⁴ and R⁵ are the same or different and each is
(1) a hydrogen atom (only for R⁴);
(2) a C₁₋₁₀ alkyl group (preferably methyl, ethyl) optionally substituted by 1 to 3 substituents selected from a C₁₋₆ alkoxy-carbonyl group, a C₁₋₆ alkoxy group and a heterocyclic group (preferably 2-thienyl);
(3) a C₃₋₁₀ cycloalkyl group optionally condensed with a benzene ring (preferably indanyl, tetrahydronaphthyl);

(4) a C₆₋₁₄ aryl group (preferably phenyl) optionally substituted by 1 to 3 substituents selected from

(4a) a halogen atom;

(4b) a C₁₋₆ alkyl group optionally substituted by a C₁₋₆ alkoxy-carbonyl group;

(4c) a C₁₋₆ alkyl-carbonyl group;

(4d) a C₁₋₆ alkylsulfonyl group; and

(4e) a C₁₋₆ alkoxy group optionally substituted by 1 to 3 halogen atoms (e.g., fluorine, chlorine, bromine, iodine);

(5) a C₇₋₁₃ aralkyl group (preferably benzyl) optionally substituted by 1 to 3 C₁₋₆ alkylsulfonyl groups;

(6) a heterocyclic group (e.g., pyridyl, isoxazolyl, pyrazolyl, thiadiazolyl, benzothiadiazolyl, oxodihydropyrazolyl, azabicyclooctyl, pyrrolidinyl) optionally substituted by 1 to 3 substituents selected from a C₁₋₆ alkyl group, a C₆₋₁₄ aryl group, a C₇₋₁₃ aralkyl group and a C₁₋₆ alkoxy-carbonyl group; or

(7) a C₁₋₆ alkoxy group (only for R⁵); or
R⁴ and R⁵ form, together with the adjacent nitrogen atom, a nitrogen-containing heterocycle (preferably pyrrolidine, piperidine, piperazine, morpholine, homopiperidine, homopiperazine, thiazolidine, dihydroindole, dihydroisoindole, tetrahydroquinoline, triazaspirodecanedione, hexahydropyradinooxazinone) optionally substituted by 1 to 3 substituents selected from
(1) a carbamoyl group;
(2) a C₁₋₆ alkyl-carbonyl group;
(3) a C₁₋₆ alkoxy-carbonyl group;
(4) a C₁₋₆ alkylsulfonyl group;
(5) a C₁₋₆ alkyl group optionally substituted by 1 to 3 substituents selected from a C₁₋₆ alkoxy group and a C₁₋₆ alkoxy-carbonyl group;
(6) a non-aromatic heterocyclic group (e.g., pyrrolidinyl);
(7) an amino group optionally mono- or di-substituted by C₁₋₆ alkyl-carbonyl group(s); and
(8) a halogen atom (e.g., fluorine, chlorine, bromine, iodine).

### [Compound B]

Of the above-mentioned [Compound A], a compound wherein X is —OH.

### [Compound C]

[5-(aminomethyl)-2-ethyl-6-isobutyl-4-(4-methylphenyl)pyridin-3-yl]acetic acid (Example 1);
[5-(aminomethyl)-2,6-diisobutyl-4-(4-methylphenyl)pyridin-3-yl]acetic acid (Example 2);
[5-(aminomethyl)-2-ethyl-4-(4-methylphenyl)-6-neopentylpyridin-3-yl]acetic acid (Example 4); or
1-{[5-(aminomethyl)-2-ethyl-4-(4-methylphenyl)-6-neopentylpyridin-3-yl]acetyl}-L-prolinamide (Example 6); or a salt thereof.

As a salt of compound (I), a pharmacologically acceptable salt is preferable. Examples of such a salt include a salt with inorganic base, a salt with organic base, a salt with inorganic acid, a salt with organic acid, a salt with basic or acidic amino acid and the like.

Preferable examples of the salt with inorganic base include alkali metal salts such as sodium salt, potassium salt and the like; alkaline earth metal salts such as calcium salt, magnesium salt and the like; aluminum salt; ammonium salt and the like.

Preferable examples of the salt with organic base-include a salt with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine and the like.

Preferable examples of the salt with inorganic acid include a salt with hydrochloric acid, hydrobromic acid, nitric acid, sulfuric acid, phosphoric acid and the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include a salt with arginine, lysin, ornithine and the like.

Preferable examples of the salt with acidic amino acid include a salt with aspartic acid, glutamic acid and the like.

Of the above-mentioned salts, the salt with inorganic acid and the salt with organic acid are preferable, hydrochloride, trifluoroacetate and the like are more preferable.

A prodrug of compound (I) is a compound that converts to compound (I) due to the reaction by enzyme, gastric acid and the like under the physiological conditions in the body; that is, a compound that converts to compound (I) by enzymatic oxidation, reduction, hydrolysis and the like, and a compound that converts to compound (I) by hydrolysis and the like by gastric acid and the like. Examples of a prodrug of compound (I) include a compound wherein an amino group of compound (I) is acylated, alkylated or phosphorylated (e.g., a compound where amino group of compound (I) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated);

a compound wherein a hydroxy group of compound (I) is acylated, alkylated, phosphorylated or borated (e.g., a compound where a hydroxy group of compound (I) is acetylated, palmitoylated, propanoylated, pivaloylated, succinylated, fumarylated, alanylated or dimethylaminomethylcarbonylated); a compound wherein a carboxyl group of compound (I) is esterified or amidated (e.g., a compound where a carboxyl group of compound (I) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. These compounds can be produced from compound (I) according to a method known per se.

A prodrug of compound (I) may be a compound that converts to compound (I) under physiological conditions as described in Development of Pharmaceutical Products, vol. 7, Molecule Design, 163-198, Hirokawa Shoten (1990).

The compound (I) may be labeled with an isotope (e.g., ³H, ¹⁴C, ³⁵S, ¹²⁵I and the like) and the like.

The compound (I) may be an anhydride or a hydrate.

The compound (I) and a prodrug thereof (hereinafter sometimes to be simply referred to as the compound of the present invention) show low toxicity and can be used as an agent for the prophylaxis or treatment of various diseases to be mentioned later for mammals (e.g., human, mouse, rat, rabbit, dog, cat, cattle, horse, swine, simian) as they are or by admixing with a pharmacologically acceptable carrier and the like to give a pharmaceutical composition.

Here, various organic or inorganic carriers conventionally used as materials for pharmaceutical preparations are used as a pharmacologically acceptable carrier, which are added as an excipient, a lubricant, a binder, a disintegrant and the like for solid preparations; and a solvent, a dissolution aid, a suspending agent, an isotonicity agent, a buffer, a soothing agent and the like for liquid preparations. Where necessary, an additive for pharmaceutical preparations such as a preservative, an antioxidant, a coloring agent, a sweetening agent and the like can be used.

Preferable examples of the excipient include lactose, sucrose, D-mannitol, D-sorbitol, starch, pregelatinized starch, dextrin, crystalline cellulose, low-substituted hydroxypropyl cellulose, sodium carboxymethylcellulose, powdered acacia, pullulan, light silicic anhydride, synthetic aluminum silicate, magnesium aluminate metasilicate and the like.

Preferable examples of the lubricant include magnesium stearate, calcium stearate, talc, colloidal silica and the like.

Preferable examples of the binder include pregelatinized starch, saccharose, gelatin, powdered acacia, methylcellulose, carboxymethylcellulose, sodium carboxymethylcellulose, crystalline cellulose, sucrose, D-mannitol, trehalose, dextrin, pullulan, hydroxypropyl cellulose, hydroxypropyl methylcellulose, polyvinylpyrrolidone and the like.

Preferable examples of the disintegrant include lactose, sucrose, starch, carboxymethylcellulose, calcium carboxymethylcellulose, sodium croscarmellose, sodium carboxymethyl starch, light silicic anhydride, low-substituted hydroxypropyl cellulose and the like.

Preferable examples of the solvent include water for injection, physiological brine, Ringer's solution, alcohol, propylene glycol, polyethylene glycol, sesame oil, corn oil, olive oil, cottonseed oil and the like.

Preferable examples of the dissolution aid include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropyl cellulose and the like; polysorbates, polyoxyethylene hydrogenated castor oil, and the like.

Preferable examples of the isotonicity agent include sodium chloride, glycerol, D-mannitol, D-sorbitol, glucose and the like.

Preferable examples of the buffer include phosphate buffer, acetate buffer, carbonate buffer, citrate buffer and the like.

Preferable examples of the soothing agent include benzyl alcohol and the like.

Preferable examples of the preservative include p-oxybenzoates, chlorobutanol, benzyl alcohol, phenethyl alcohol, dehydroacetic acid, sorbic acid and the like.

Preferable examples of the antioxidant include sulfite, ascorbate and the like.

Preferable examples of the coloring agent include water-soluble edible tar pigments (e.g., foodcolors such as Food Color Red Nos. 2 and 3, Food Color Yellow Nos. 4 and 5, Food Color Blue Nos. 1 and 2 and the like), water insoluble lake pigments (e.g., aluminum salt of the aforementioned water-soluble edible tar pigment), natural pigments (e.g., beta carotene, chlorophil, red iron oxide) and the like.

Preferable examples of the sweetening agent include saccharin sodium, dipotassium glycyrrhizinate, aspartame, stevia and the like.

The dosage form of the aforementioned pharmaceutical composition is, for example, an oral agent such as tablets (inclusive of sublingual tablets and orally disintegrable tablets), capsules (inclusive of soft capsules and microcapsules), granules, powders, troches, syrups, emulsions, suspensions and the like; or a parenteral agent such as injections (e.g., subcutaneous injections, intravenous injections, intramuscular injections, intraperitoneal injections, drip infusions), external agents (e.g., transdermal preparations, ointments), suppositories (e.g., rectal suppositories, vaginal suppositories), pellets, nasal preparations, pulmonary preparations (inhalations), ophthalmic preparations and the like. These may be administered safely via an oral or parenteral route.

These agents may be controlled-release preparations such as rapid-release preparations and sustained-release preparations (e.g., sustained-release microcapsules).

The pharmaceutical composition can be produced according to a method conventionally used in the field of pharmaceutical preparation, such as the method described in Japan Pharmacopoeia and the like.

While the content of the compound of the present invention in the pharmaceutical composition varies depending on the dosage form, dose of the compound of the present invention and the like, it is, for example, about 0.1-100 wt %.

Where necessary, the aforementioned oral agents may be coated with a coating base for the purpose of masking taste, enteric property or sustained release.

Examples of the coating base include a sugar-coating base, a water-soluble film coating base, an enteric film coating base, a sustained-release film coating base and the like.

As the sugar-coating base, sucrose may be used, if necessary, along with one or more species selected from talc, precipitated calcium carbonate, gelatin, powdered acacia, pullulan, carnauba wax and the like.

As the water-soluble film coating base, for example, cellulose polymers such as hydroxypropyl cellulose, hydroxypropyl methylcellulose, hydroxyethylcellulose, methylhydroxyethylcellulose and the like; synthetic polymers such as polyvinyl acetal diethylaminoacetate, aminoalkyl methacrylate copolymer E [Eudragit E, trade name, Roehm Pharma], polyvinylpyrrolidone and the like; polysaccharides such as pullulan and the like; and the like are used.

As the enteric film coating base, for example, cellulose polymers such as hydroxypropyl methylcellulose phthalate, hydroxypropyl methylcellulose acetate succinate, carboxymethylethylcellulose, cellulose acetate phthalate and the like; acrylic acid polymers such as methacrylic acid copolymer L [Eudragit L, trade name, Roehm Pharma], methacrylic acid copolymer LD [Eudragit L-30D55, trade name, Roehm Pharma], methacrylic acid copolymer S [Eudragit S, trade name, Roehm Pharma] and the like; natural products such as shellac and the like; and the like are used.

As the sustained-release film coating base, for example, cellulose polymers such as ethylcellulose and the like; acrylic acid polymers such as aminoalkyl methacrylate copolymer RS [Eudragit R^{S}, trade name, Roehm Pharma], ethyl acrylate-methyl methacrylate copolymer suspension [Eudragit NE, trade name, Roehm Pharma] and the like; and the like are used.

Two or more kinds of the above-mentioned coating bases may be mixed in an appropriate ratio for use. In addition, a light shielding agent such as titanium oxide, ferric oxide and the like may be used during coating.

The compound of the present invention shows low toxicity (e.g., acute toxicity, chronic toxicity, genetic toxicity, reproductive toxicity, vascular toxicity, carcinogenic), causes fewer side effects and can be used as an agent for the prophylaxis or treatment or diagnosis of various diseases for mammals (e.g., human, cattle, horse, dog, cat, simian, mouse, rat, especially human).

The compound of the present invention has a superior peptidase inhibitory activity and can suppress peptidase-caused degradation of a physiologically active substance such as peptide hormones, cytokines, neurotransmitters and the like.

Examples of the peptide hormones include glucagon-like peptide-1 (GLP-1), glucagon-like peptide-2 (GLP-2), GIP, growth hormone release hormone (GHRH) and the like.

Examples of the cytokines include chemokine such as RANTES and the like.

Examples of the neurotransmitters include neuropeptide Y and the like.

Examples of the peptidases include EC 3.4.11.1 (Leucyl aminopeptidase), EC 3.4.11.2 (Membrane alanine aminopeptidase), EC 3.4.11.3 (Cystinyl aminopeptidase), EC 3.4.11.4 (Tripeptide aminopeptidase), EC 3.4.11.5 (Prolyl aminopeptidase), EC 3.4.11.6 (Aminopeptidase B), EC 3.4.11.7 (Glutamyl aminopeptidase), EC 3.4.11.9 (Xaa-Pro aminopeptidase), EC 3.4.11.10 (Bacterial leucyl aminopeptidase), EC 3.4.11.13 (Clostridial aminopeptidase), EC 3.4.11.14 (Cytosol alanyl aminopeptidase), EC 3.4.11.15 (Lysyl aminopeptidase), EC 3.4.11.16 (Xaa-Trp aminopeptidase), EC 3.4.11.17 (Tryptophanyl aminopeptidase), EC 3.4.11.18 (Methionyl aminopeptidase), EC 3.4.11.19 (D-stereospecific aminopeptidase), EC 3.4.11.20 (Aminopeptidase Ey), EC 3.4.11.21 (Aspartyl aminopeptidase), EC 3.4.11.22 (Aminopeptidase I), EC 3.4.13.3 (Xaa-His dipeptidase), EC 3.4.13.4 (Xaa-Arg dipeptidase), EC 3.4.13.5 (Xaa-methyl-His dipeptidase), EC 3.4.13.7 (Glu-Glu dipeptidase), EC 3.4.13.9 (Xaa-Pro dipeptidase), EC 3.4.13.12 (Met-Xaa dipeptidase), EC 3.4.13.17 (Non-stereospecific dipeptidase), EC 3.4.13.18 (Cytosol nonspecific dipeptidase), EC 3.4.13.19 (Membrane dipeptidase), EC 3.4.13.20 (Beta-Ala-His dipeptidase), EC 3.4.14.1 (Dipeptidyl-peptidase I), EC 3.4.14.2 (Dipeptidyl-peptidase II), EC 3.4.14.4 (Dipeptidyl-peptidase III), EC 3.4.14.5 (Dipeptidyl-peptidase IV), EC 3.4.14.6 (Dipeptidyl-dipeptidase), EC 3.4.14.9 (Tripeptidyl-peptidase I), EC 3.4.14.10 (Tripeptidyl-peptidase II), EC 3.4.14.11 (Xaa-Pro dipeptidyl-peptidase) and the like as classified by International Union of Biochemistry and Molecular Biology. As the peptidase, FAP_{α}, DPP8, DPP9 and the like can be also mentioned.

Of these, EC 3.4.14.1, EC 3.4.14.2, EC 3.4.14.4, EC 3.4.14.5, EC 3.4.14.6, EC 3.4.14.9, EC 3.4.14.10 and EC 3.4.14.11 are preferable. Especially preferred is EC 3.4.14.5 (Dipeptidyl-peptidase IV).

The compound of the present invention may concurrently have a glucagon antagonistic action or a CETP (Cholesteryl ester transfer protein) inhibitory action in addition to a peptidase inhibitory action. When the compound of the present invention concurrently has these actions, the compound of the present invention is more effective as an agent for the prophylaxis or treatment of diabetes (e.g., type 1 diabetes, type 2 diabetes, gestational diabetes mellitus, slowly progressive insulin dependent diabetes mellitus (SPIDDM), LADA (Latent Autoimmune Diabetes in Adults), insulinopenic diabetes, obese diabetes) and hyperlipidemia (e.g., hypertriglyceridemia, hypercholesteremia, hypoHDLemia, postprandial hyperlipidemia).

The compound of the present invention can be used as an agent for the prophylaxis or treatment of diabetes (e.g., type 1 diabetes, type 2 diabetes, gestational diabetes, slowly progressive insulin dependent diabetes mellitus (SPIDDM), LADA (Latent Autoimmune Diabetes in Adults), insulinopenic diabetes, obese diabetes); an agent for the prophylaxis or treatment of hyperlipidemia (e.g., hypertriglyceridemia, hypercholesterolemia, hypoHDLemia, postprandial hyperlipidemia); an agent for the prophylaxis or treatment of arteriosclerosis; an agent for the prophylaxis or treatment of impaired glucose tolerance [IGT]; an insulin secretagogue; and an agent for preventing progress of impaired glucose tolerance into diabetes.

For diagnostic criteria of diabetes, Japan Diabetes Society, ADA (American Diabetes Association) and WHO reported new diagnostic criteria.

According to these reports, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

According to the above-mentioned reports, impaired glucose tolerance is a condition showing a fasting blood glucose level (glucose concentration of intravenous plasma) of less than 126 mg/dl and a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose).

According to the report of WHO, among the IFG (Impaired Fasting Glucose), a condition showing a 75 g oral glucose tolerance test 2 h level (glucose concentration of intravenous plasma) of less than 140 mg/dl is called IFG (Impaired Fasting Glycemia).

The compound of the present invention can be also used as an agent for the prophylaxis or treatment of diabetes, borderline type, impaired glucose tolerance, IFG (Impaired Fasting Glucose) and IFG (Impaired Fasting Glycemia), as determined according to the above-mentioned diagnostic criteria. Moreover, the compound of the present invention can prevent progress of borderline type, impaired glucose tolerance, IFG (Impaired Fasting Glucose) or IFG (Impaired Fasting Glycemia) into diabetes.

The compound of the present invention can be also used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infection, inferior limb infection), diabetic gangrene, xerostomia, hypacusis, cerebrovascular disorder, peripheral blood circulation disorder], obesity, osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, hypertension, polycystic ovary syndrome, kidney disease (e.g., diabetic nephropathy, glomerular nephritis, glomerulosclerosis, nephrotic syndrome, hypertensive nephrosclerosis, end stage kidney disease), muscular dystrophy, myocardial infarction, angina pectoris, cerebrovascular accident (e.g., cerebral infarction, cerebral apoplexy), Alzheimer's disease, Parkinson's syndrome, anxiety, dementia, schizophrenia, insulin resistance syndrome, Syndrome X, metabolic syndrome, hyperinsulinemia, hyperinsulinemia-induced sensory disorder, tumor (e.g., leukemia, breast cancer, prostatic cancer, skin cancer), irritable bowel syndrome, acute or chronic diarrhea, inflammatory diseases (e.g., chronic rheumatoid arthritis, spondylitis deformans, osteoarthritis, lumbago, gout, postoperative or traumatic inflammation, tumentia, neuralgia, pharyngolaryngitis, cystitis, hepatitis (inclusive of nonalcoholic steatohepatitis), pneumonia, pancreatitis, enteritis, inflammatory bowel diseases (including inflammatory disease of large intestine), ulcerative colitis, gastric mucosal injury (inclusive of gastric mucosal injury caused by aspirin)), small intestine mucous membrane trauma, malabsorption, testis function disorder, visceral obesity syndrome and the like.

The compound of the present invention can be also used for decreasing visceral fat, suppressing visceral fat accumulation, improving glycometabolism, improving lipid metabolism, suppressing production of oxidized LDL, improving lipoprotein metabolism, improving coronary artery metabolism, prophylaxis and treatment of cardiovascular complications, prophylaxis and treatment of heart failure complications, lowering blood remnant, prophylaxis and treatment of anovulation, prophylaxis and treatment of hypertrichosis, prophylaxis and treatment of hyperandrogenemia, improving pancreatic (β cell) function, regeneration of pancreatic (β cell), promotion of pancreatic (β cell) regeneration, appetite control and the like.

The compound of the present invention can be also used for secondary prophylaxis and prevention of progression of the above-mentioned various diseases (e.g., cardiovascular event such as myocardial infarction and the like).

The compound of the present invention is a glucose dependent insulin secretagogue that selectively promotes insulin secretion in hyperglycemic patients (e.g., patients showing fasting blood glucose level of not less than 126 mg/dl or 75 g oral glucose tolerance test (75 g OGTT) 2 h level of not less than 140 mg/dl). Therefore, the compound of the present invention is useful as a safe agent for the prophylaxis or treatment of diabetes with a low risk of vascular complications, hypoglycemia induction and the like caused by insulin.

The compound of the present invention is also useful as a therapeutic agent for diabetes with sulfonylurea secondary failure and affords a superior insulin secretion effect and a hypoglycemic effect for diabetic patients for whom sulfonylurea compounds and fast-acting insulin secretagogues fail to provide an insulin secretion effect, and therefore, fail to provide a sufficient hypoglycemic effect.

As the sulfonylurea compound here, a compound having a sulfonylurea skeleton or a derivative thereof, such as tolbutamide, glibenclamide, gliclazide, chlorpropamide, tolazamide, acetohexamide, glyclopyramide, glimepiride, glipizide, glybuzole and the like can be mentioned.

As the fast-acting insulin secretagogue, a compound that promotes insulin secretion from pancreatic β cell in the same manner as a sulfonylurea compound, though it does not have a sulfonylurea skeleton, such as glinide compounds (e.g., repaglinide, senaglinide, nateglide, mitiglinide, a calcium salt hydrate thereof), and the like, can be mentioned.

While the dose of the compound of the present invention varies depending on the administration subject, administration route, target disease, condition and the like, the compound of the present invention is generally given in a single dose of about 0.01-100 mg/kg body weight, preferably 0.05-30 mg/kg body weight, more preferably 0.1-10 mg/kg body weight, in the case of, for example, oral administration to adult diabetic patients. This dose is desirably given 1 to 3 times a day.

The compound of the present invention can be used in combination with drugs such as a therapeutic agent for diabetes, a therapeutic agent for diabetic complications, an antihyperlipemic agent, an antihypertensive agent, an antiobestic agent, a diuretic, a chemotherapeutic agent, an immunotherapeutic agent, an antithrombotic agent, a therapeutic agent of osteoporosis, an antidementia agent, an agent for improving erectile dysfunction, a therapeutic agent for incontinentia or pollakiuria, a therapeutic agent for dysurea and the like (hereinafter to be referred to as a combination drug). In this case, the timing of administration of the compound of the present invention and a combination drug is not limited. These may be simultaneously administered to an administration subject or administered in a staggered manner. Moreover, the compound of the present invention and a combination drug may be administered as two kinds of preparations each containing an active ingredient, or may be administered as a single preparation containing both active ingredients.

The dose of the combination drug can be determined as appropriate based on the dose clinically employed. The proportion of the compound of the present invention and combination drug can be appropriately determined depending on the administration subject, administration route, target disease, condition, combination and the like. When, for example, the administration subject is human, a combination drug is used in an amount of 0.01-100 parts by weight per 1 part by weight of the compound of the present invention.

As the therapeutic agent for diabetes, insulin preparations (e.g., animal insulin preparations extracted from the pancreas of bovine and pig; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (preferably hydrochloride), rosiglitazone or a salt thereof (preferably maleate), Reglixane (JTT-501), GI-262570, Netoglitazone (MCC-555), DRF-2593, KRP-297, R-119702, Rivoglitazone (CS-011), FK-614, compounds described in WO99/58510 (e.g., (E)-4-[4-(5-methyl-2-phenyl-4-oxazolylmethoxy)benzyloxyimino]-4-phenylbutyric acid), compounds described in WO01/38325, Tesaglitazar (AZ-242), Ragaglitazar (N,N-622), Muraglitazar (BMS-298585), ONO-5816, Edaglitazone (BM-13-1258), LM-4156, MBX-102, Naveglitazar (LY-519818), MX-6054, LY-510929, Balaglitazone (N,N-2344), T-131 or a salt thereof, THR-0921), PPARγ agonist, PPARγ antagonist, PPARγ/α dual agonist, α-glucosidase inhibitors (e.g., voglibose, acarbose, miglitol, emiglitate), biguanides (e.g., phenformin, metformin, buformin or salts thereof (e.g., hydrochloride, fumarate, succinate)), insulin secretagogues [sulfonylurea (e.g., tolbutamide, glibenclamide, gliclazide, chlorpropamide, tolazamide, acetohexamide, glyclopyramide, glimepiride, glipizide, glybuzole), repaglinide, senaglinide, nateglide, mitiglinide or calcium salt hydrate thereof], GPR40 agonist, GLP-1 receptor agonists [e.g., GLP-1, GLP-1MR, Liraglutide (N,N-2211), Exenatide (AC-2993, exendin-4), Exenatide LAR, BIM-51077, Aib(8,35)hGLP-1(7,37)NH₂, CJC-1131], amylin agonists (e.g., pramlintide), phosphotyrosine phosphatase inhibitors (e.g., sodium vanadate), dipeptidyl peptidase IV inhibitors (e.g., NVP-DPP-728, PT-100, P32/98, Vidagliption (LAF-237), P93/01, TS-021, Sitagliptin (MK-0431), Saxagliptin (BMS-477118), T-6666), β3 agonist (e.g., AJ-9677, AZ40140), gluconeogenesis inhibitors (e.g., glycogen phosphorylase inhibitor, glucose-6-phosphatase inhibitor, glucagon antagonist), SGLT (sodium-glucose cotransporter) inhibitors (e.g., T-1095), 11β-hydroxysteroid dehydrogenase inhibitors (e.g., BVT-3498), adiponectin or agonist thereof, IKK inhibitors (e.g., AS-2868), leptin resistance improving drugs, somatostatin receptor agonists (compounds described in WO01/25228, WO03/42204, WO98/44921, WO98/45285, WO99/22735), glucokinase activators (e.g., Ro-28-1675) and the like can be mentioned.

Examples of the therapeutic agent for diabetic complications include aldose reductase inhibitors (e.g., Tolrestat, Epalrestat, Zenarestat, Zopolrestat, Minalrestat, Fidarestat (SNK-860), CT-112, Ranirestat), neurotrophic factors and increasing drugs thereof (e.g., NGF, NT-3, BDNF, neurotrophin production-secretion promoters described in WO01/14372 (e.g., 4-(4-chlorophenyl)-2-(2-methyl-1-imidazolyl)-5-[3-(2-methylphenoxy)propyl]oxazole)), neuranagenesis stimulators (e.g., Y-128), PKC inhibitors (e.g., ruboxistaurin mesylate; LY-333531), AGE inhibitors (e.g., ALT946, pimagedine, pyratoxanthine, N-phenacylthiazolium bromide (ALT766), ALT-711, EXO-226, Pyridorin, Pyridoxamine), reactive oxygen scavengers (e.g., thioctic acid), cerebral vasodilators (e.g., tiapride, mexiletine), somatostatin receptor agonists (e.g., BIM23190) and apoptosis signal regulating kinase-1 (ASK-1) inhibitors.

Examples of the antihyperlipemic agent include statin compounds which are HMG-CoA reductase inhibitor (e.g., pravastatin, simvastatin, lovastatin, atorvastatin, fluvastatin, rosuvastatin, pitavastatin and salts thereof (e.g., sodium salt, calcium salt)), squalene synthase inhibitors (e.g., compounds described in WO97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid), fibrate compounds (e.g., bezafibrate, clofibrate, simfibrate, clinofibrate), ACAT inhibitors (e.g., Avasimibe, Eflucimibe), anion exchange resins (e.g., colestyramine), probucol, nicotinic acid drugs (e.g., nicomol, niceritrol), ethyl icosapentate, plant sterols (e.g., soysterol, γ-oryzanol) and the like.

Examples of the antihypertensive agent include angiotensin converting enzyme inhibitors (e.g., captopril, enalapril, delapril), angiotensin II receptor antagonists (e.g., candesartan cilexetil, losartan, eprosartan, valsartan, telmisartan, irbesartan, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid), calcium antagonists (e.g., manidipine, nifedipine, amlodipine, efonidipine, nicardipine), potassium channel openers (e.g., levcromakalim, L-27152, AL 0671, NIP-121), Clonidine and the like.

Examples of the antiobestic agent include antiobestic agents acting on the central nervous system (e.g., Dexfenfluramine, fenfluramine, phentermine, Sibutramine, amfepramone, dexamphetamine, Mazindol, phenylpropanolamine, clobenzorex; MCH receptor antagonists (e.g., SB-568849; SNAP-7941; compounds encompassed in WO01/82925 and WO01/87834); neuropeptide Y antagonists (e.g., CP-422935); cannabinoid receptor antagonists (e.g., SR-141716, SR-147778); ghrelin antagonist; 11β-hydroxysteroid dehydrogenase inhibitors (e.g., BVT-3498)), pancreatic lipase inhibitors (e.g., orlistat, ATL-962), β3 agonists (e.g., AJ-9677, AZ40140), peptidic anorexiants (e.g., leptin, CNTF (Ciliary Neurotropic Factor)), cholecystokinin agonists (e.g., lintitript, FPL-15849), feeding deterrent (e.g., P-57), GPR⁴⁰ antagonist and the like.

Examples of the diuretic include xanthine derivatives (e.g., sodium salicylate and theobromine, calcium salicylate and theobromine), thiazide preparations (e.g., ethiazide, cyclopenthiazide, trichloromethyazide, hydrochlorothiazide, hydroflumethiazide, benzylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlortalidone, mefruside, indapamide), azosemide, isosorbide, etacrynic acid, piretanide, bumetanide, furosemide and the like.

Examples of the chemotherapeutic agent include alkylation agents (e.g., cyclophosphamide, ifosfamide), metabolic antagonists (e.g., methotrexate, 5-fluorouracil or its derivative), anti-cancer antibiotics (e.g., mitomycin, adriamycin), plant-derived anti-cancer agents (e.g., vincristin, vindesine, taxol), cisplatin, carboplatin, etoposide and the like. Of these, furtulon and neofurtulon, which are 5-fluorouracil derivatives, and the like are preferable.

Examples of the immunotherapeutic agent include microorganism or bacterial components (e.g., muramyl dipeptide derivative, picibanil), polysaccharides having immunity potentiating activity (e.g., lentinan, sizofuran, krestin), cytokines obtained by genetic engineering techniques (e.g., interferon, interleukin (IL)), colony stimulating factors (e.g., granulocyte colony stimulating factor, erythropoietin) and the like, with preference given to interleukins such as IL-1, IL-2, IL-12 and the like.

Examples of the antithrombotic agent include heparin (e.g., heparin sodium, heparin calcium, dalteparin sodium), warfarin (e.g., warfarin potassium), anti-thrombin drugs (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like.

Examples of the therapeutic agent of osteoporosis include alfacalcidol, calcitriol, elcatonin, calcitonin salmon, estriol, ipriflavone, pamidronate disodium, alendronate sodium hydrate, incadronate disodium, risedronate disodium and the like.

Examples of the antidementia agent include tacrine, donepezil, rivastigmine, galanthamine and the like.

Examples of the agent for improving erectile dysfunction include apomorphine, sildenafil citrate and the like.

Examples of the therapeutic agent for incontinentia or pollakiuria include flavoxate hydrochloride, oxybutynin hydrochloride, propiverine hydrochloride and the like.

Examples of the therapeutic agent for dysurea include acetylcholine esterase inhibitors (e.g., distigmine) and the like.

Furthermore, drugs having a cachexia-improving action established in animal models and clinical situations, such as cyclooxygenase inhibitors (e.g., Indometacin), Progesterone derivatives (e.g., Megesterol acetate), glucosteroid (e.g., dexamethasone), metoclopramide agents, tetrahydrocannabinol agents, fat metabolism improving agents (e.g., eicosapentaenoic acid), growth hormones, IGF-1, or antibodies to a cachexia-induced factor such as TNF-α, LIF, IL-6, Oncostatin M and the like, can be used in combination with the compound of the present invention.

The combination drug is preferably an insulin preparation, an insulin sensitizer, an α-glucosidase inhibitor, a biguanide, an insulin secretagogue (preferably sulfonylurea) and the like.

Two or more of the above-mentioned combination drugs can be used in combination in an appropriate ratio. Preferable combinations in the case of using two or more combination drugs are, for example, as shown in the following.
1) an insulin secretagogue (preferably sulfonylurea) and an α-glucosidase inhibitor;
2) an insulin secretagogue (preferably sulfonylurea) and a biguanide;
3) an insulin secretagogue (preferably sulfonylurea), a biguanide and an α-glucosidase inhibitor;
4) an insulin sensitizer and an α-glucosidase inhibitor;
5) an insulin sensitizer and a biguanide;
6) an insulin sensitizer, a biguanide and an α-glucosidase inhibitor.

By combining the compound of the present invention and a combination drug, a superior effect such as

(1) the dose of the compound of the present invention and/or combination drug can be reduced as compared to single administration of the compound of the present invention or a combination drug,
(2) a sustained treatment effect can be designed by selecting a combination drug having different action and mechanism from the compound of the present invention,
(3) a synergistic effect can be afforded by a combined use of the compound of the present invention and a combination drug, and the like, can be achieved.

When the compound of the present invention is used in combination with a combination drug, the amount thereof can be reduced within a safe range in consideration of adverse effect of these agents. Particularly, the dose of an insulin sensitizer, an insulin secretagogue (preferably sulfonylurea) and a biguanide can be reduced as compared with the normal dose. Therefore, an adverse effect, which may be caused by these agents, can be prevented safely. In addition, the dose of the therapeutic agent for diabetic complications, antihyperlipemic agent and antihypertensive agent can be reduced whereby an adverse effect, which may be caused by these agents, can be prevented effectively.

Hereinafter the production methods of the compound of the present invention are explained.

The compound of the present invention can be produced according to a method known per se, such as a method to be described in detail in the following, or an analogous method thereto.

Compounds 1 to 14 in the following formulas may form a salt, and as such a salt, for example, salts similar to the salt of compound (I) can be mentioned.

While the compounds obtained in respective steps of the following formulas can be used for the next reaction in the form of a reaction mixture or a crude product, they can also be easily isolated and purified from the reaction mixture by a known separation and purification means, such as concentration, concentration under reduced pressure, solvent extraction, crystallization, recrystallization, phase transfer, chromatography and the like.

When the compounds in the following formulas are commercially available, commercially available products can also be used as they are.

In each of the following reactions, when the starting compound has an amino group, a carboxyl group or a hydroxy group as a substituent, a protecting group generally used in peptide chemistry and the like may be introduced into these groups. By eliminating the protecting group as necessary after the reaction, the objective compound can be obtained.

In the present specification, as the amino-protecting group, for example, formyl group, C₁₋₆ alkyl-carbonyl group, C₁₋₆ alkoxy-carbonyl group, benzoyl group, C₇₋₁₀ aralkyl-carbonyl group (e.g., benzylcarbonyl), C₇₋₁₄ aralkyloxy-carbonyl group (e.g., benzyloxycarbonyl, 9-fluorenylmethoxycarbonyl), trityl group, phthaloyl group, N,N-dimethylaminomethylene group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkoxy group and nitro group.

As the carboxyl-protecting group, for example, C₁₋₆ alkyl group, C₇₋₁₁ aralkyl group (e.g., benzyl), phenyl group, trityl group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned.

As the hydroxy-protecting group, for example, C₁₋₆ alkyl group, phenyl group, trityl group, C₇₋₁₀ aralkyl group (e.g., benzyl), formyl group, C₁₋₆ alkyl-carbonyl group, benzoyl group, C₇₋₁₀ aralkyl-carbonyl group (e.g., benzylcarbonyl), 2-tetrahydropyranyl group, 2-tetrahydrofuranyl group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkyl group, C₁₋₆ alkoxy group and nitro group.

For elimination of the above-mentioned protecting group, a method known per se, for example, a method described in Protective Groups in Organic Synthesis, John Wiley and Sons (1980) and the like can be mentioned. For example, employed is a method using acid, base, UV light, hydrazine, phenyl hydrazine, sodium N-methyldithlocarbamate, tetrabutylammonium fluoride, palladium acetate, trialkylsilyl halide (e.g., trimethylsilyl iodide, trimethylsilyl bromide and the like) and the like, reduction and the like.

Compound (1-a), which is a compound of the formula (I) wherein X is a hydroxyl group, can be produced according to the following Scheme 1 or a method analogous thereto.

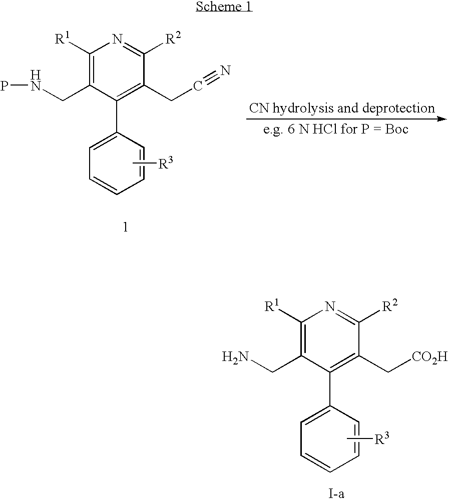

wherein the symbols in the formula are as defined above.

The amino-protecting group for P is preferably a C₁₋₆ alkoxy-carbonyl group (preferably Boc(tert-butoxycarbonyl) group)), a C₇₋₁₄ aralkyloxy-carbonyl group (preferably Cbz(benxyloxycarbonyl) group, Fmoc(9-fluorenylmethoxycarbonyl group)) and the like.

In this method, the cyano group of compound 1 is hydrolyzed, and the amino-protecting group is simultaneously or subsequently eliminated to give compound (1-a).

The hydrolysis can be generally carried out in the presence of an acid or base.

As the acid, for example, mineral acids (e.g., hydrochloric acid, hydrobromide acid, sulfuric acid, phosphoric acid), carboxylic acids (e.g., formic acid, acetic acid, propionic acid) and the like can be mentioned. Of these, hydrochloric acid, sulfuric acid and the like are preferable.

As the base, for example, alkali metal salts such as lithium hydroxide, potassium hydroxide, sodium hydroxide, potassium carbonate, sodium carbonate, potassium hydrogencarbonate, sodium hydrogencarbonate and the like; alkaline earth metal salts such as calcium hydroxide, barium hydroxide and the like; amines such as trimethylamine, triethylamine, N,N-diisopropylethylamine, N-methylmorpholine and the like; and the like can be mentioned. Of these, potassium hydroxide, sodium hydroxide and the like are preferable.

The amount of the acid or base to be used is generally 0.01 to 100 mol, preferably 0.1 to 50 mol, per 1 mol of compound 1.

The hydrolysis is generally carried out in a solvent that does not adversely affect the reaction. As such a solvent, for example, alcohols such as methanol, ethanol, propanol, isopropanol, butanol, isobutanol, tert-butanol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, dimethoxyethane and the like; amides such as N,N-dimethylformamide, N,N-dimethylacetamide and the like; sulfoxides such as dimethyl sulfoxide and the like; water and the like can be mentioned.

Two or more kinds of these solvents may be used in a mixture at an appropriate ratio.

The reaction temperature is generally 0° C. to 150° C., preferably 10° C. to 100° C.

While the reaction time varies depending on the acid or base reagent and solvent to be used, it is generally 0.1 to 100 hrs, preferably 0.1 to 10 hrs.

The amino-protecting group can be eliminated according to a method known per se.

Compound 1 to be used as a starting compound in the aforementioned Scheme 1 can be produced according to the following Scheme 2 or a method analogous thereto.

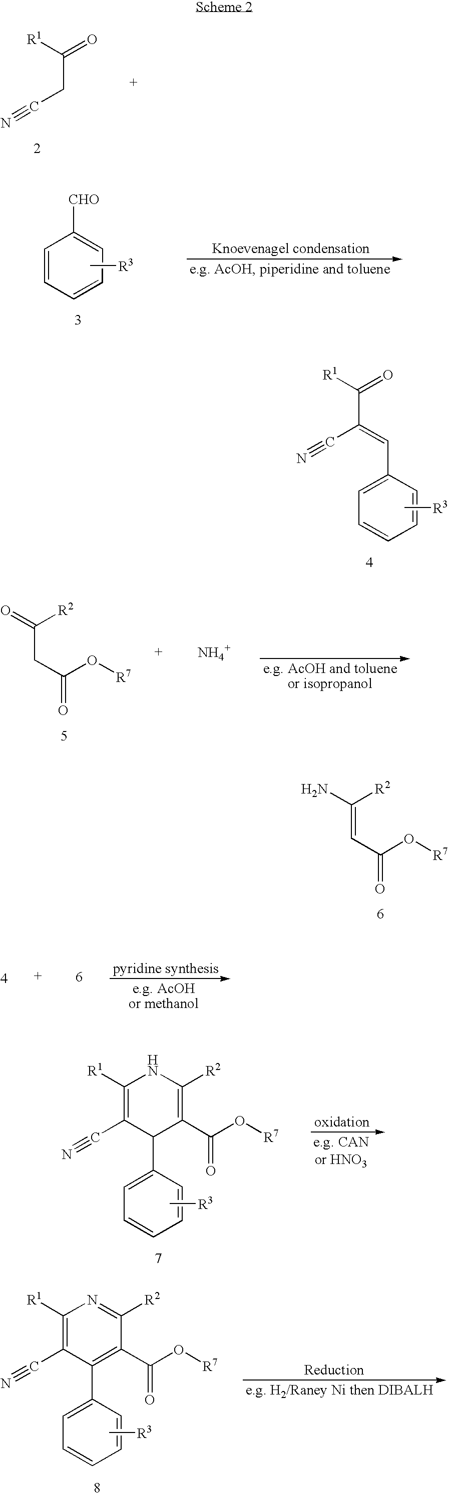

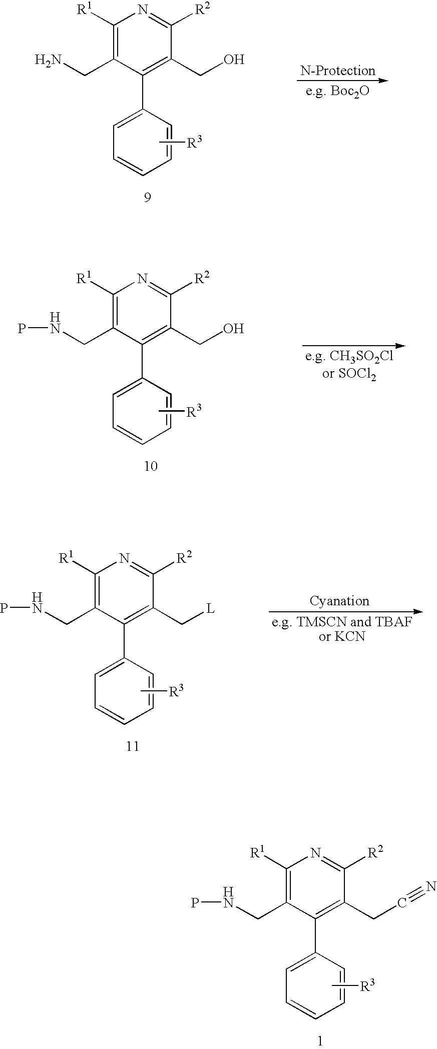

wherein R⁷ is an optionally substituted C₁₋₁₀ alkyl group, L is a leaving group (e.g., a substituted sulfonyloxy group (e.g., methanesulfonyloxy group, p-toluenesulfonyloxy group), a halogen atom (e.g., chlorine, bromine)), and other symbols are as defined above.

As the optionally substituted C₁₋₁₀ alkyl group for R⁷, those exemplified for the aforementioned R⁶ can be mentioned.

Compound 1 can be produced, for example, by cyanation of compound 11 using a cyanating agent. As the cyanating agent to be used here, conventional cyanating agents, such as potassium cyanide, trimethylsilane carbonitrile (TMSCN) and the like can be mentioned. When potassium cyanide is used as a cyanating agent, reaction efficiency can be improved by the addition of tetrabutylammonium bromide and the like, and when trimethylsilane carbonitrile is used, reaction efficiency can be improved by the addition of tetrabutylammonium fluoride (TBAF).

Compound 11 can be produced, for example, by converting the hydroxyl group of compound 10 to a leaving group. Conversion to a leaving group can be carried out according to a conventional method, for example, by reacting with methanesulfonyl chloride in the presence of a suitable base, or by reacting with thionyl chloride in the presence of a suitable base, and the like. As the suitable base used for conversion to a leaving group, for example, N,N-diisopropylethylamine (DIEA), triethylamine (TEA), pyridine, N,N-dimethylaniline and the like can be mentioned.

Compound 10 can be produced, for example, by protecting the amino group of compound 9. Protection of the amino group can be carried out according to a method known per se.

Compound 9 can be produced, for example, by subjecting compound 8 to a reduction reaction, thereby converting the 5-position substituent (i.e., cyano group) and the 3-position substituent (i.e., substituted oxycarbonyl group) to an aminomethyl group and a hydroxymethyl group, respectively. The reduction reaction of the cyano group and that of the substituted oxycarbonyl group can be carried out sequentially or simultaneously. When the reduction reactions are sequentially carried out, either of the reduction reactions may be carried out first and, where necessary, the intermediate obtained upon completion of one reduction reaction may be isolated and purified and then the intermediate may be subjected to the other reduction reaction. Such a reduction reaction is carried out according to a conventional method in the presence of a reducing agent in a solvent that does not adversely affect the reaction.

As the reducing agent, for example, metal hydride compounds such as sodium bis(2-methoxyethoxy)aluminum hydride, diisobutylaluminum hydride (DIBALH) and the like; metal hydride complex compounds such as sodium borohydride, sodium cyanoborohydride, lithium aluminum hydride, sodium aluminum hydride and the like; and the like can be mentioned.

The amount of the reduction agent to be used is generally 0.1 to 20 mol per 1 mol of compound 8.

As the solvent that does not adversely affect the reaction, for example, alcohols such as methanol, ethanol, propanol, isopropanol, butanol, isobutanol, tert-butanol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, dimethoxyethane and the like; esters such as methyl acetate, ethyl acetate, n-butyl acetate, tert-butyl acetate and the like; amides such as N,N-dimethylformamide, N,N-dimethylacetamide, N-methylpyrrolidone and the like are used. These solvents may be used in a mixture of two or more kinds thereof at an appropriate ratio.

The reaction temperature is generally −70° C. to 150° C., preferably −20° C. to 100° C.

The reaction time is generally 0.1 to 100 hrs, preferably 0.1 to 40 hrs.

The reduction reaction of the cyano group can also be carried out in a solvent that does not adversely affect the reaction in the presence of a metal-catalyst (e.g., palladium-carbon, palladium black, palladium chloride, platinum oxide, platinum black, platinum-palladium, Raney-nickel, Raney-cobalt) and a hydrogen source.

The amount of the metal catalyst to be used is generally 0.001 to 1000 mol, preferably 0.01 to 100 mol, per 1 mol of compound 8.

As the hydrogen source, for example, hydrogen gas, formic acid, amine salt of formic acid, phosphinate, hydrazine and the like can be mentioned. As the solvent that does not adversely affect the reaction, for example, methanol, tetrahydrofuran, N,N-dimethylacetamide and the like can be mentioned.

This reaction may be carried out, where necessary, in the presence of ammonia (e.g., aqueous ammonia, ammonia-methanol). Reaction in the presence of ammonia suppresses side reactions and compound 9 can be produced in a high yield.

Compound 8 can be produced, for example, by oxidation of compound 7. The oxidation reaction is carried out according to a conventional method in the presence of an oxidant (e.g., dilute nitric acid, cerium ammonium nitrate (CAN)) in a solvent that does not adversely affect the reaction (e.g., dioxane, acetone).

Compound 7 can be produced, for example, from compound 4 and compound 6, according to a method known per se, such as a Hantzch's pyridine synthetic method described in “Shin Jikken Kagaku Kouza (The Chemical Society of Japan ed.), Vol. 14, Synthesis and Reaction of Organic Compound IV, Maruzen (1978), page 2057, or a method analogous thereto.

Compound 4 can be produced by a method known per se, for example, by subjecting compound 2 and compound 3 to a known Knoevenagel condensation.

Compound 6 can be produced by a reaction of compound 5 with ammonia or ammonium salt, according to a method known per se, such as methods described in Synthesis, (1999), vol. 11, p. 1951-1960; Journal of Chemical Society Perkin Transactions 1, (2002), p. 1663-1671 and the like or methods analogous thereto.

The aforementioned compound 2, compound 3 and compound 5 can be produced by a method known per se.

Compound (1-b), which is a compound of the formula (I) wherein X is —OR⁸ [R⁸ is an optionally substituted hydrocarbon group or an optionally substituted heterocyclic group], can be produced according to the following Scheme 3 or a method analogous thereto.

As the optionally substituted hydrocarbon group and optionally substituted heterocyclic group for R⁸, those exemplified for the aforementioned R⁶ can be mentioned, respectively.

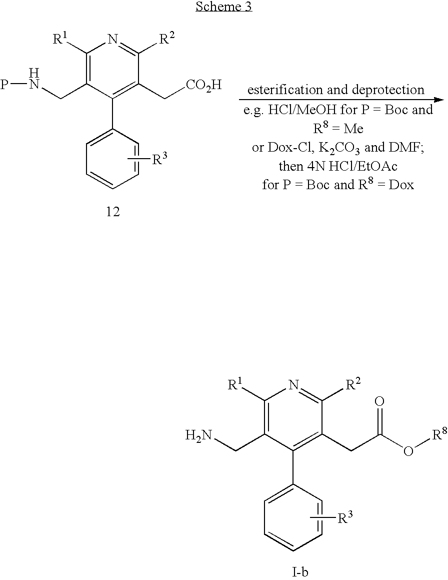

wherein Dox is (5-methyl-2-oxo-1,3-dioxol-4-yl)methyl group, and other symbols are as defined above.

In this method, compound 12 is esterified and, where necessary, the amino-protecting group is eliminated simultaneously or subsequently to give compound (I-b).

For esterification, a method known per se, such as esterification with an alcohol (R⁸—OH), esterification with an O-alkylating agent (R⁸-L) and the like can be mentioned.

The esterification with an alcohol is carried out according to a conventional method by reacting compound 12 with an alcohol in the presence of an acid catalyst or dehydrating agent. While this reaction is generally carried out in a solvent that does not adversely affect the reaction, the alcohol itself may be used as a solvent.

As the acid catalyst, acids generally used as an acid catalyst in condensation, such as hydrochloric acid, sulfuric acid, p-toluenesulfonic acid, boron fluoride etherate and the like, can be mentioned.

The amount of the acid catalyst to be used is preferably about 0.05 to about 50 mol per 1 mol of compound 12. As the dehydrating agent, a reagent that activates compound 12 (e.g., dicyclohexylcarbodiimide (DCC), trifluoroacetic anhydride), a reagent that activates alcohols (e.g., combination of an organophosphorus compound (e.g., triphenylphosphine) and an electrophilic agent (e.g., diethyl azodicarboxylate)), and the like can be mentioned.

The amount of the dehydrating agent to be used is preferably about 1 to about 50 mol per 1 mol of compound 12.

As the solvent that does not adversely affect the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide (DMF) and the like; sulfoxides such as dimethyl sulfoxide and the like, and the like can be mentioned. These solvents may be mixed at an appropriate ratio.

The reaction temperature is generally −30° C. to 150° C.

The reaction time is generally 0.5 to 20 hrs.

The esterification with an O-alkylating agent is carried out according to a conventional method, for example, using an O-alkylating agent in the presence of a base in a solvent that does not adversely affect the reaction.

As the base, bases generally used for O-alkylation of a carboxyl group, such as amines (e.g., triethylamine, N-methylmorpholine, N,N-dimethylaniline); alkali metal salts (e.g., sodium hydrogencarbonate, sodium carbonate, potassium carbonate); and the like can be mentioned.

The amount of each of the O-alkylating agent and base to be used is preferably about 1 to about 50 mol per 1 mol of compound 12.

As the solvent that does not adversely affect the reaction, for example, ethers such as tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethyl sulfoxide and the like; and the like can be mentioned. These solvents may be used in a mixture at an appropriate ratio.

The reaction temperature is generally −30° C. to 100° C.

The reaction time is generally 0.5 to 20 hrs.

The amino-protecting group can be eliminated according to a method known per se.

Compound (I-c), which is a compound of the formula (I) wherein X is —NR⁴R⁵ can be produced according to the following Scheme 4 or a method analogous thereto.

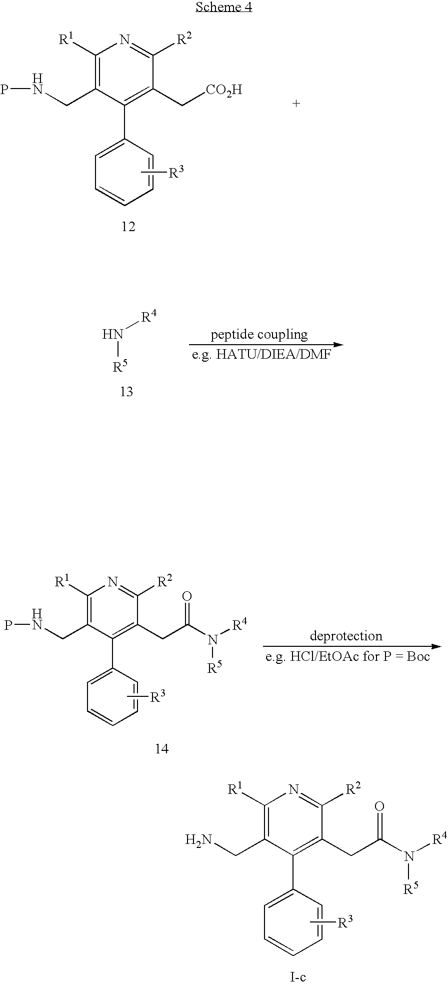

wherein each symbol is as defined above.

In this method, compound 12 is condensed with compound 13, and then the amino-protecting group is eliminated to give compound (I-c).

The condensation is carried out according to a conventional method, for example, conventional peptide coupling method. As such a method, for example, direct condensation of compound 12 with compound 13 using a condensing agent, reaction of a reactive derivative of compound 12 with compound 13 and the like can be mentioned.

As the condensing agent, for example, carbodiimide condensing reagents such as dicyclohexylcarbodiimide (DCC), diisopropylcarbodiimide (DIPC), 1-ethyl-3-(3-dimethylaminopropyl)carbodiimide (EDC), hydrochlorides thereof and the like; phosphoric acid condensing reagents such as diethyl cyanophosphate, diphenylphosphoryl azide and the like; carbonyldiimidazole, 2-chloro-1,3-dimethylimidazolium tetrafluoroborate, O-(7-azabenzotriazol-1-yl)-1,1,3,3-tetramethyluronium hexafluorophosphate (HATU) and the like can be mentioned.

As the solvent to be used for the reaction using a condensing agent, for example, amides such as N,N-dimethylformamide, N,N-dimethylacetamide, N-methylpyrrolidone and the like; sulfoxides such as dimethyl sulfoxide and the like; halogenated hydrocarbons such as chloroform, dichloromethane and the like; aromatic hydrocarbons such as benzene, toluene and the like; ethers such as tetrahydrofuran, dioxane, diethyl ether, dimethoxyethane and the like; esters such as methyl acetate, ethyl acetate and the like; nitrites such as acetonitrile, propionitrile and the like; water; and the like can be mentioned. These solvents may be used in a mixture at an appropriate ratio.

The amount of compound 13 to be used is generally 1 to 10 mol, preferably 1 to 3 mol, per 1 mol of compound 12.

The amount of the condensing agent to be used is generally 0.1 to 10 mol, preferably 0.3 to 3 mol, per 1 mol of compound 12.

When a carbodiimide condensing reagent is used as the condensing agent, reaction efficiency can be improved by using, as necessary, a suitable condensation promoter (e.g., 1-hydroxy-7-azabenzotriazole, 1-hydroxybenzotriazole, N-hydroxysuccinimide, N-hydroxyphthalimide). In addition, when HATU or a phosphoric acid condensing reagent is used as the condensing reagent, reaction efficiency can be improved by using an organic amine base such as triethylamine, N,N-diisopropylethylamine and the like.

The amount of each of the above-mentioned condensation promoter and organic amine base to be used is generally 0.1 to 10 mol, preferably 0.3 to 3 mol, per 1 mol of compound 12.

The reaction temperature is generally −30° C. to 120° C., preferably −10° C. to 100° C.

The reaction time is generally 0.5 to 60 hrs.

As the reactive derivative of compound 12, for example, an acid anhydride, an acid halide (e.g., an acid chloride, an acid bromide), an imidazolide, a mixed acid anhydride (e.g., an anhydride with methyl carbonate, ethyl carbonate, isobutyl carbonate), and the like can be mentioned.

When, for example, an acid anhydride or an acid halide is used, the reaction is generally carried out in the presence of a base in a solvent that does not adversely affect the reaction.

As the base, for example, amines such as triethylamine, pyridine, N-methylmorpholine, N,N-dimethylaniline, 4-dimethylaminopyridine and the like; alkali metal salts such as lithium hydroxide, sodium hydroxide, potassium hydroxide, sodium hydrogencarbonate, sodium carbonate, potassium carbonate and the like, and the like can be mentioned.

As the solvent that does not adversely affect the reaction, for example, amides such as N,N-dimethylformamide, N,N-dimethylacetamide, N-methylpyrrolidone and the like; sulfoxides such as dimethyl sulfoxide and the like; halogenated hydrocarbons such as chloroform, dichloromethane and the like; aromatic hydrocarbons such as benzene, toluene and the like; ethers such as tetrahydrofuran, dioxane, diethyl ether, dimethoxyethane and the like; esters such as methyl acetate, ethyl acetate and the like; nitriles such as acetonitrile, propionitrile and the like; water; and the like can be mentioned. These solvents may be used in a mixture at an appropriate ratio.

When the above-mentioned amides are used as a solvent that does not adversely affect the reaction, the reaction can also be carried out in the absence of a base.

The amount of compound 13 to be used is generally 1 to 10 mol, preferably 1 to 5 mol, per 1 mol of compound 12.

The amount of the base to be used is generally 1 to 10 mol, preferably 1 to 5 mol, per 1 mol of compound 12.

The reaction temperature is generally −30° C. to 100° C., preferably −10° C. to 100° C.

The reaction time is generally 0.5 to 30 hrs.

When a mixed acid anhydride is used, compound 12 is reacted with a chlorocarbonate (e.g., methyl chlorocarbonate, ethyl chlorocarbonate, isobutyl chlorocarbonate) in the presence of a base and the resulting compound is reacted with compound 13.

As the base to be used here, for example, those exemplified in the above for a base to be used for the reaction of an acid anhydride or acid halide of compound 12 with compound 13 and the like can be mentioned.

The amount of compound 13 to be used is generally 1 to 10 mol, preferably 1 to 5 mol, per 1 mol of compound 12.

The amount of the base to be used is generally 1 to 10 mol, preferably 1 to 3 mol, per 1 mol of compound 12.

The reaction temperature is generally −30° C. to 120° C., preferably −10° C. to 100° C.

The reaction time is generally 0.5 to 20 hrs.

When an imidazolide is used, a corresponding imidazolide is obtained from compound 12 and, for example, N,N′-carbonyldiimidazole (CDI), which is then reacted with compound 13.

The amount of compound 13 to be used is generally 1 to 10 mol, preferably 1 to 5 mol, per 1 mol of compound 12.

The reaction temperature is generally −30° C. to 120° C., preferably −10° C. to 100° C.

The reaction time is generally 0.5 to 20 hrs.

The amino-protecting group can be eliminated according to a method known per se.

The compound (I) thus obtained can be isolated and purified by a known separation and purification means, such as concentration, concentration under reduced pressure, solvent extraction, crystallization, recrystallization, phase transfer, chromatography and the like.

When compound (I) is obtained as a free compound, it can be converted to the object salt according to a method known per se or a method analogous thereto, and when it is obtained as a salt, it can be converted to a free compound or the object salt according to a method known per se or a method analogous thereto.

When compound (I) contains an optical isomer, a stereoisomer, a positional isomer or a rotational isomer, these are also encompassed in compound (I), and can be obtained as a single product according to a synthetic method and separation method known per se. For example, when compound (I) has an optical isomer, an optical isomer resolved from this compound is also encompassed in compound (I).

The optical isomer can be produced according to a method known per se. To be specific, an optically active synthetic intermediate is used, or the final racemate product is subjected to optical resolution according to a conventional method to give an optical isomer.

The method of optical resolution may be a method known per se, such as a fractional recrystallization method, a chiral column method, a diastereomer method and the like.

### 1) Fractional Recrystallization Method

A salt of a racemate with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine) is formed, which is separated by a fractional recrystallization method, and a free optical isomer is obtained by a neutralization step where desired.

### 2) Chiral Column Method

A racemate or a salt thereof is applied to a column for separation of an optical isomer (chiral column) to allow separation. In the case of a liquid chromatography, for example, a mixture of optical isomers is applied to a chiral column such as ENANTIO-OVM (manufactured by Tosoh Corporation), CHIRAL series (manufactured by Daicel Chemical Industries, Ltd.) and the like, and developed with water, various buffers (e.g., phosphate buffer) and organic solvents (e.g., ethanol, methanol, isopropanol, acetonitrile, trifluoroacetic acid, diethylamine) solely or in admixture to separate the optical isomer. In the case of a gas chromatography, for example, a chiral column such as CP-Chirasil-DeX CB (manufactured by GL Sciences Inc.) and the like is used to allow separation.

### 3) Diastereomer Method

A racemic mixture is prepared into a diastereomeric mixture by chemical reaction with an optically active reagent, which is prepared into a single substance by a typical separation means (e.g., fractional recrystallization, chromatography method) and the like, and subjected to a chemical treatment such as hydrolysis and the like to separate the optically active reagent moiety, whereby the optical isomer is obtained. For example, when compound (I) contains a hydroxy group or a primary or secondary amino group in a molecule, the compound and an optically active organic acid (e.g., MTPA [α-methoxy-α-(trifluoromethyl)phenylacetic acid], (−)-menthoxyacetic acid) and the like are subjected to condensation to give an ester form diastereomer thereof or an amide form diastereomer thereof, respectively. When compound (I) has a carboxyl group, this compound and an optically active amine or an optically alcohol are subjected to condensation to give an amide form diastereomer thereof or an ester form diastereomer thereof, respectively. The separated diastereomer is converted to the optical isomer of the original compound by acidic hydrolysis or basic hydrolysis.

The compound (I) may be in the form of a crystal.

The crystal of compound (I) (hereinafter sometimes to be referred to as crystal of the present invention) can be produced by crystallization of compound (I) according to a crystallization method known per se.

Examples of the crystallization method include crystallization from a solution, crystallization from vapor, crystallization from a molten form and the like.

The “crystallization from a solution” is typically a method including shifting a non-saturated state to supersaturated state by varying factors involved in solubility of compounds (solvent composition, pH, temperature, ionic strength, redox state etc.) or the amount of solvent. To be specific, for example, concentration method, annealing method, reaction method (diffusion method, electrolysis method), hydrothermal growth method, fusing agent method and the like can be mentioned. Examples of the solvent to be used include aromatic hydrocarbons (e.g., benzene, toluene, xylene), halogenated hydrocarbons (e.g., dichloromethane, chloroform), saturated hydrocarbons (e.g., hexane, heptane, cyclohexane), ethers (e.g., diethyl ether, diisopropyl ether, tetrahydrofuran, dioxane), nitriles (e.g., acetonitrile), ketones (e.g., acetone), sulfoxides (e.g., dimethyl sulfoxide), acid amides (e.g., N,N-dimethylformamide), esters (e.g., ethyl acetate), alcohols (e.g., methanol, ethanol, isopropyl alcohol), water and the like.

These solvents are used alone or in combination of two or more at a suitable ratio (e.g., 1:1 to 1:100 (volume ratio)).

The “crystallization from vapor” is, for example, vaporization method (sealed tube method, gas stream method), gas phase reaction method, chemical transportation method and the like.

The “crystallization from a molten form” is, for example, normal freezing method (Czockralski method, temperature gradient method, Bridgman method), zone melting method (zone leveling method, floating zone method), special growth method (VLS method, liquid phase epitaxy method) and the like.

Preferable examples of the crystallization method include a method including dissolving compound (I) or a salt thereof in a suitable solvent (e.g., alcohols such as methanol, ethanol etc.) at a temperature of 20 to 120° C. and cooling the resulting solution to a temperature not higher than the temperature of dissolution (e.g., 0 to 50° C., preferably 0 to 20° C.) and the like.

The thus-obtained crystals of the present invention can be isolated by, for example, filtration and the like.

In the present specification, the melting point refers to that measured using, for example, micromelting point measuring apparatus (Yanako, MP-500D or Buchi, B-545) or DSC (differential scanning calorimetry) device (SEIKO, EXSTAR6000) and the like.

In general, melting points vary depending on measurement apparatuses, measurement conditions and the like. The crystal in the present specification may show a different melting point described in the present specification, as long as it is within general error range.

The crystal of the present invention is superior in physicochemical properties (e.g., melting point, solubility, stability) and biological properties (e.g., pharmacokinetics (absorption, distribution, metabolism, excretion), efficacy expression), and is extremely useful as a pharmaceutical agent.

### EXAMPLES

The present invention is explained in more detail by referring to the following Reference Examples, Examples, Experimental Examples and Formulation Examples, which are not to be construed as limitative. The present invention can be modified within the range that does not deviate from the scope of the invention.

Abbreviations in the Reference Examples and Examples mean the following:

s: singlet, d: doublet, t: triplet, q: quartet, m: multiplet, J: coupling constant.

In the Examples, room temperature means a temperature of 10 to 30° C., and % means percent by weight, unless otherwise specified.

In the following Reference Examples and Examples, mass spectrum (MS) was measured by Electron Spray Ionization method using Waters Corporation ZQ, ZMP or SHIMADZU CORPORATION LCMS-2010A. For purification by silica gel column chromatography, flash chromatography (mobile phase: solvent selected from hexane, ethyl acetate and methanol or a mixed solvent thereof) was employed. For purification by HPLC, Gilson, Inc., high through-put purification system (YMC Combiprep Hydrosphere C18, S-5 μm, 50×20 mm; mobile phase: gradient elution from 2% acetonitrile, 98% water and 0.1% trifluoroacetic acid to 95% acetonitrile, 5% water and 0.1% trifluoroacetic acid) was employed.

TABLE 1

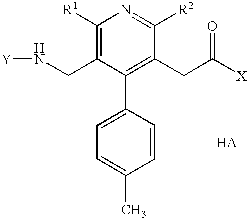

| Ref. Ex. | R¹ | R² | —X | Y— | HA |
|---|---|---|---|---|---|
| 1 | isobutyl | methyl | —OH | 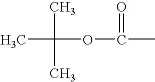 | none |
| 2 | neopentyl | methyl | —OH | 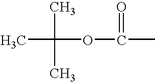 | none |
| 3 | isobutyl | isobutyl | —OH | 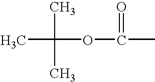 | none |
| 4 | neopentyl | ethyl | —OH | 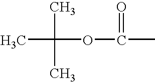 | none |
| 5 | isobutyl | methyl | 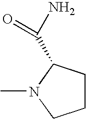 | H— | 2HCl |
| 6 | isobutyl | methyl | 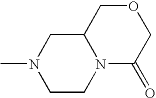 | H— | 2HCl |
| 7 | isobutyl | methyl | 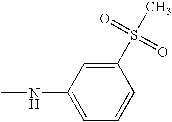 | H— | 2HCl |

### Reference Example 1 [5-{[(tert-butoxycarbonyl)amino]methyl}-6-isobutyl-2-methyl-4-(4-methylphenyl)pyridin-3-yl]acetic acid

### Step A. methyl 5-cyano-6-isobutyl-2-methyl-4-(4-methylphenyl)-1,4-dihydropyridine-3-carboxylate

A mixture of 5-methyl-3-oxohexanenitrile (5.0 g, 40 mmol) prepared by a method similar to EP 135252 A2 (Ex. Y), p-tolualdehyde (4.8 g, 40 mmol), piperidine (0.34 g, 4.0 mmol), acetic acid (0.48 g, 8.0 mmol) and toluene (200 mL) was heated under reflux for 12 hrs using a Dean-Stark trap. The reaction mixture was allowed to cool to room temperature, washed with saturated brine, dried over anhydrous magnesium sulfate and concentrated under reduced pressure. The obtained residue was dissolved in methanol (50 mL), methyl 3-aminocrotonate (4.6 g, 40 mmol) was added, and the mixture was heated under reflux for 6 hrs. The reaction mixture was concentrated under reduced pressure, and the residue was purified by silica gel column chromatography to give the title compound (7.45 g, yield 57%) as colorless crystals.

melting point: 171° C.

### Step B. methyl 5-cyano-6-isobutyl-2-methyl-4-(4-methylphenyl)nicotinate

To an ice-cooled solution of methyl 5-cyano-6-isobutyl-2-methyl-4-(4-methylphenyl)-1,4-dihydropyridine-3-carboxylate (75.5 g, 0.23 mol) in acetone (500 mL) was added dropwise a solution of cerium ammonium nitrate (319 g, 0.58 mol) in water (300 mL). The obtained mixture was stirred under ice-cooling for 1 hr and concentrated under reduced pressure. The residue was partitioned between ethyl acetate and water, and the organic layer was washed successively with saturated aqueous sodium hydrogencarbonate and saturated brine, dried over anhydrous magnesium sulfate and concentrated under reduced pressure. The residue was crystallized from hexane to give the title compound (69.4 g, yield 93%) as a white powder.

MS 323(M+1).

### Step C. methyl 5-(aminomethyl)-6-isobutyl-2-methyl-4-(4-methylphenyl)nicotinate

A mixture of methyl 5-cyano-6-isobutyl-2-methyl-4-(4-methylphenyl)nicotinate (1.00 g, 3.10 mmol), Raney-nickel (4 mL), 25% aqueous ammonia (6 mL), tetrahydrofuran (15 mL) and methanol (45 mL) was stirred in a sealed tube at room temperature under a hydrogen atmosphere of 0.3 to 0.5 MPa for 6 hrs. The reaction mixture was filtered, and the filtrate was concentrated under reduced pressure. The residue was partitioned between ethyl acetate and 10% aqueous potassium carbonate solution. The organic layer was washed with saturated brine, dried over anhydrous magnesium sulfate and concentrated under reduced pressure. The residue was purified by silica gel column chromatography to give the title compound (0.97 g, yield 95%) as pale-yellow crystals.

MS 327(M+1).

### Step D. tert-butyl {[5-(hydroxymethyl)-2-isobutyl-6-methyl-4-(4-methylphenyl)pyridin-3-yl]methyl}carbamate

A solution of methyl 5-(aminomethyl)-6-isobutyl-2-methyl-4-(4-methylphenyl)nicotinate (9.3 g, 29 mmol) in toluene (150 mL) was cooled to −78° C., and 1 M diisobutylaluminum hydride toluene solution (100 mL, 100 mmol) was added dropwise over 30 min. The obtained mixture was allowed to warm and acetone (10 mL) and sodium sulfate decahydrate (40 g) were added at 0° C. The reaction mixture was stirred overnight at room temperature and insoluble materials were filtered off and washed with ethyl acetate. The filtrate and the washing solution were combined, and 1N aqueous sodium hydroxide solution (30 mL, 30 mmol) and di-tert-butyl dicarbonate (6.9 mL, 30 mmol) were added. The mixture was stirred at room temperature for 30 min. The reaction mixture was washed successively with water and saturated brine, dried over anhydrous magnesium sulfate and concentrated under reduced pressure. The residue was purified by silica gel column chromatography to give the title compound (8.5 g, yield 75%) as colorless crystals.

MS 399(M+1).

### Step E. tert-butyl {[5-(cyanomethyl)-2-isobutyl-6-methyl-4-(4-methylphenyl)pyridin-3-yl]methyl}carbamate

To an ice-cooled mixture of tert-butyl {[5-(hydroxymethyl)-2-isobutyl-6-methyl-4-(4-methylphenyl)pyridin-3-yl]methyl}carbamate (17.4 g, 43 mmol), triethylamine (15 mL, 108 mmol) and tetrahydrofuran (150 mL) was added dropwise methanesulfonyl chloride (4.0 mL, 52 mmol), and the mixture was stirred at 0° C. for 30 min. Water was added to the reaction mixture, and the mixture was extracted with ethyl acetate. The extract was washed with saturated brine, dried over anhydrous magnesium sulfate and concentrated under reduced pressure to give [5-{[(tert-butoxycarbonyl)amino]methyl}-6-isobutyl-2-methyl-4-(4-methylphenyl)pyridin-3-yl]methyl methanesulfonate as a crude product (20 g). The crude product (20 g) was dissolved in acetonitrile (300 mL), and trimethylsilane carbonitrile (6.7 mL, 50 mmol) and then 1 M tetrabutylammonium fluoride tetrahydrofuran solution (50 mL, 50 mmol) were successively added. The obtained mixture was stirred at room temperature for 1 hr and concentrated under reduced pressure. Water was added to the residue and the mixture was extracted with ethyl acetate. The extract was washed with saturated brine, dried over anhydrous magnesium sulfate and concentrated under reduced pressure. The residue was washed with a mixture of hexane and diethyl ether to give the title compound (15.6 g, yield 89%) as a white powder.

MS 408(M+1).

### Step F. [5-{[(tert-butoxycarbonyl)amino]methyl}-6-isobutyl-2-methyl-4-(4-methylphenyl)pyridin-3-yl]acetic acid

tert-Butyl {[5-(cyanomethyl)-2-isobutyl-6-methyl-4-(4-methylphenyl)pyridin-3-yl]methyl}carbamate (14.5 g, 36 mmol) was suspended in 6N hydrochloric acid (150 mL) and the suspension was stirred at 90° C. for 20 hrs. The reaction mixture was allowed to cool to room temperature, and washed with diethyl ether. The aqueous layer was alkalified (pH 8) with 8N aqueous sodium hydroxide solution, and ethyl acetate (200 mL) and di-tert-butyl dicarbonate (10 mL, 44 mmol) were added. The mixture was stirred at room temperature for 1 hr. The reaction mixture was neutralized with hydrochloric acid and partitioned. The aqueous layer was extracted with ethyl acetate and the organic layer and the extract were combined. The mixture was washed with saturated brine, dried over anhydrous magnesium sulfate and concentrated under reduced pressure to give the title compound (14.0 g, yield 92%) as a white powder.

MS 427(M+1).

### Reference Example 2 [5-{[(tert-butoxycarbonyl)amino]methyl}-2-methyl-4-(4-methylphenyl)-6-neopentylpyridin-3-yl]acetic acid

### Step A. 2-(3,3-dimethylbutanoyl)-3-(4-methylphenyl)acrylonitrile

A mixture of 5,5-dimethyl-3-oxohexanenitrile (302 g, 2.2 mol) prepared by a method similar to EP 135252 A2 (Ex. V), p-tolualdehyde (256 mL, 2.2 mol), piperidine (22 mL, 0.22 mol), acetic acid (25 mL, 0.43 mol) and toluene (1.4 L) was heated under reflux for 8 hrs using a Dean-Stark trap. The reaction mixture was allowed to cool to room temperature, washed with saturated brine, and dried over anhydrous magnesium sulfate. The solvent was evaporated under reduced pressure and the residue was recrystallized from hexane-toluene to give the title compound (390 g, yield 75%) as a white powder.

¹H-NMR (CDCl₃) δ: 1.10 (9H, s), 2.44 (3H, s), 2.81 (2H, s), 7.31 (2H, d, J=8.1 Hz), 7.92 (2H, d, J=8.3 Hz), 8.13 (1H, s).

### Step B. tert-butyl 5-cyano-2-methyl-4-(4-methylphenyl)-6-neopentyl-1,4-dihydropyridine-3-carboxylate

A mixture of 2-(3,3-dimethylbutanoyl)-3-(4-methylphenyl)acrylonitrile (495 g, 2.1 mol), tert-butyl 3-aminobut-2-enoate (354 g, 2.3 mol) and acetic acid (2.5 L) was stirred at 80° C. for 30 min. The reaction mixture was ice-cooled and the precipitated crystals were collected by filtration, washed with 75% aqueous ethanol and dried to give the title compound (611 g, yield 78%) as colorless crystals.

melting point: 202° C.

### Step C. tert-butyl 5-cyano-2-methyl-4-(4-methylphenyl)-6-neopentylnicotinate

The title compound (134 g, yield 89%) was obtained as a white powder from tert-butyl 5-cyano-2-methyl-4-(4-methylphenyl)-6-neopentyl-1,4-dihydropyridine-3-carboxylate (152 g, 0.40 mol) by a method similar to Step B of Reference Example 1.

MS 379(M+1).

### Step D. 5-cyano-2-methyl-4-(4-methylphenyl)-6-neopentylnicotinic acid

A solution of tert-butyl 5-cyano-2-methyl-4-(4-methylphenyl)-6-neopentylnicotinate (20 g, 53 mmol) in trifluoroacetic acid (50 mL) was stirred at 50° C. for 12 hrs. The reaction mixture was concentrated under reduced pressure and the residue was crystallized from hexane-ethyl acetate to give the title compound (12.1 g, yield 71%) as a white powder.

MS 323(M+1).

### Step E. 5-(hydroxymethyl)-6-methyl-4-(4-methylphenyl)-2-neopentylnicotinonitrile

To an ice-cooled mixture of 5-cyano-2-methyl-4-(4-methylphenyl)-6-neopentylnicotinic acid (0.83 g, 2.6 mmol), N,N-dimethylformamide (0.02 mL) and tetrahydrofuran (20 mL) was added dropwise oxalyl chloride (0.39 g, 3.1 mmol). The obtained mixture was stirred at 5° C. for 30 min and concentrated under reduced pressure. The residue was dissolved in a mixture of tetrahydrofuran (10 mL) and 1,2-dimethoxyethane (10 mL), and ice-cooled. To the ice-cooled solution was added sodium tetrahydroborate (0.34 g, 9.0 mmol), and the mixture was stirred. To the mixture was added dropwise methanol (3 mL), and the mixture was stirred at room temperature for 30 min and partitioned between ethyl acetate and water. The organic layer was washed with saturated brine, dried over anhydrous magnesium sulfate and concentrated under reduced pressure. The residue was purified by silica gel column chromatography to give the title compound (0.69 g, yield 87%) as colorless crystals.

MS 309(M+1).

### Step F. tert-butyl {[5-(hydroxymethyl)-6-methyl-4-(4-methylphenyl)-2-neopentylpyridin-3-yl]methyl}carbamate

A mixture of 5-(hydroxymethyl)-6-methyl-4-(4-methylphenyl)-2-neopentylnicotinonitrile (0.61 g, 2.0 mmol), Raney-nickel (2 mL), 25% aqueous ammonia (2 mL) and methanol (50 mL) was stirred at room temperature for 1 hr in a sealed tube under a hydrogen atmosphere at 0.3-0.5 MPa. The reaction mixture was filtered, and the filtrate was concentrated under reduced pressure. The residue was dissolved in tetrahydrofuran (100 mL), to which di-tert-butyl dicarbonate (0.50 mL, 2.2 mmol) was added, and the mixture was stirred at room temperature for 1 hr. The reaction mixture was concentrated under reduced pressure, and the residue was purified by silica gel column chromatography and recrystallized from diisopropyl ether-ethyl acetate to give the title compound (0.67 g, yield 82%) as a white powder.

MS 413(M+1).

### Step G. tert-butyl {[5-(cyanomethyl)-6-methyl-4-(4-methylphenyl)-2-neopentylpyridin-3-yl]methyl}carbamate

The title compound (5.7 g, yield 93%) was obtained as colorless crystals from tert-butyl {[5-(hydroxymethyl)-6-methyl-4-(4-methylphenyl)-2-neopentylpyridin-3-yl]methyl}carbamate (6.0 g, 15 mmol) by a method similar to Step E of Reference Example 1.

MS 422(M+1).

### Step H. [5-{[(tert-butoxycarbonyl)amino]methyl}-2-methyl-4-(4-methylphenyl)-6-neopentylpyridin-3-yl]acetic acid

The title compound (13.0 g, yield 80%) was obtained as a white powder from tert-butyl {[5-(cyanomethyl)-6-methyl-4-(4-methylphenyl)-2-neopentylpyridin-3-yl]methyl}carbamate (15.5 g, 37 mmol) by a method similar to Step F of Reference Example 1.

MS 441(M+1).

### Reference Example 3 [5-{[(tert-butoxycarbonyl)amino]methyl}-2,6-diisobutyl-4-(4-methylphenyl)pyridin-3-yl]acetic acid

### Step A. methyl 5-methyl-3-oxohexanoate

Under a nitrogen atmosphere, to a solution of dimethyl carbonate (6.3 g, 70 mmol) in 1,4-dioxane (15 mL) was added sodium hydride (2.2 g, 55 mmol), and the mixture was heated under reflux. A solution of 4-methyl-2-pentanone (2.5 g, 25 mmol) in 1,4-dioxane (5 mL) was added dropwise to the suspension, and the mixture was heated under reflux for 3 hrs. The reaction mixture was poured into ice water, and the mixture was washed with hexane, neutralized with 1N hydrochloric acid and extracted with diethyl ether. The extract was washed with saturated brine, dried over anhydrous magnesium sulfate and concentrated under reduced pressure to give the title compound (3.9 g, yield 98%) as an orange oil.

¹H-NMR (CDCl₃) δ: 0.94 (6H, d, J=6.6 Hz), 2.10-2.25 (1H, m), 2.42 (2H, d, J=7.0 Hz), 3.44 (2H, s), 3.74 (3H, s).

### Step B. methyl 3-amino-5-methylhex-2-enoate

A mixture of methyl 5-methyl-3-oxohexanoate (4.0 g, 25 mmol), ammonium acetate (9.8 g, 25 mmol), acetic acid (1.5 mL, 25 mmol) and toluene (200 mL) was heated under reflux for 12 hrs using a Dean-Stark trap. The reaction mixture was allowed to cool to room temperature, washed with saturated brine, dried over anhydrous magnesium sulfate and concentrated under reduced pressure. The residue was purified by silica gel column chromatography to give the title compound (3.0 g, yield 75%) as a pale-yellow oil.

¹H-NMR (CDCl₃) δ: 0.94 (6H, d, J=6.4 Hz), 1.80-1.95 (1H, m), 1.95-2.00 (2H, m), 3.65 (3H, s), 4.52 (1H, s).

### Step C. methyl 5-cyano-2,6-diisobutyl-4-(4-methylphenyl)nicotinate

Methyl 5-cyano-2,6-diisobutyl-4-(4-methylphenyl)-1,4-dihydropyridine-3-carboxylate was obtained as a crude product from 5-methyl-3-oxohexanenitrile (2.5 g, 20 mmol), p-tolualdehyde (2.4 g, 20 mmol) and methyl 3-amino-5-methylhex-2-enoate (3.1 g, 20 mmol) by a method similar to Step A of Reference Example 1. The title compound (4.8 g, yield 65%) was obtained as a white powder from the crude product by a method similar to Step B of Reference Example 1.

¹H-NMR (CDCl₃) δ: 0.93 (6H, d, J=6.8 Hz), 1.00 (6H, d, J=6.8 Hz), 2.17-2.34 (2H, m), 2.40 (3H, s), 2.73 (2H, d, J=7.4 Hz), 2.97 (2H, d, J=7.2 Hz), 3.57 (3H, s), 7.27 (4H, s).

### Step D. methyl 5-{[(tert-butoxycarbonyl)amino]methyl}-2,6-diisobutyl-4-(4-methylphenyl)nicotinate

Methyl 5-(aminomethyl)-2,6-diisobutyl-4-(4-methylphenyl)nicotinate was obtained as a crude product from methyl 5-cyano-2,6-diisobutyl-4-(4-methylphenyl)nicotinate (4.8 g, 13 mmol) by a method similar to Step C of Reference Example 1. To a solution of the crude product in tetrahydrofuran (60 mL) was added di-tert-butyl dicarbonate (3.4 g, 16 mmol) at room temperature, and the mixture was stirred for 30 min and concentrated under reduced pressure. The residue was purified by silica gel column chromatography to give the title compound (5.8 g, yield 95%) as a white powder.

MS 469(M+1).

### Step E. tert-butyl {[5-(hydroxymethyl)-2,6-diisobutyl-4-(4-methylphenyl)pyridin-3-yl]methyl}carbamate

A solution of methyl 5-{[(tert-butoxycarbonyl)amino]methyl}-2,6-diisobutyl-4-(4-methylphenyl)nicotinate (5.4 g, 12 mmol) in toluene (50 mL) was cooled to −78° C., and 1.5 M diisobutylaluminum hydride toluene solution (35 mL, 52 mmol) was added dropwise over 30 min. The obtained mixture was stirred at −78° C. for 30 min, allowed to warm to 0° C. and stirred for 30 min. To the reaction mixture was added methanol (1 mL) and the mixture was stirred for 15 min. Sodium sulfate decahydrate (17 g) was further added and the mixture was stirred for 1 hr. Insoluble materials were filtered off and washed with ethyl acetate. The filtrate and the washing solution were combined, and the mixture was concentrated under reduced pressure. The residue was purified by silica gel column chromatography to give the title compound (2.5 g, yield 49%).

MS 441(M+1).

### Step F. tert-butyl {[5-(cyanomethyl)-2,6-diisobutyl-4-(4-methylphenyl)pyridin-3-yl]methyl}carbamate

The title compound (2.4 g, yield 94%) was obtained as a white powder from tert-butyl {[5-(hydroxymethyl)-2,6-diisobutyl-4-(4-methylphenyl)pyridin-3-yl]methyl}carbamate (2.5 g, 5.7 mmol) by a method similar to Step E of Reference Example 1.

MS 450(M+1).

### Step G. [5-{[(tert-butoxycarbonyl)amino]methyl}-2,6-diisobutyl-4-(4-methylphenyl)pyridin-3-yl]acetic acid

The title compound (1.7 g, yield 67%) was obtained as a white powder from tert-butyl {[5-(cyanomethyl)-2,6-diisobutyl-4-(4-methylphenyl)pyridin-3-yl]methyl}carbamate (2.4 g, 5.3 mmol) by a method similar to Step F of Reference Example 1.

MS 469(M+1).

### Reference Example 4 [5-{[(tert-butoxycarbonyl)amino]methyl}-2-ethyl-4-(4-methylphenyl)-6-neopentylpyridin-3-yl]acetic acid

### Step A. methyl 3-aminopent-2-enoate

A mixture of methyl 3-oxopentanoate (3.3 g, 25 mmol), ammonium acetate (9.8 g, 127 mmol), acetic acid (1.45 mL, 25 mmol) and toluene (200 mL) was heated under reflux for 12 hrs using a Dean-Stark trap. The reaction mixture was allowed to cool to room temperature, washed with saturated brine, dried over anhydrous magnesium sulfate and concentrated under reduced pressure to give the title compound as a crude product (2.5 g).

### Step B. methyl 5-cyano-2-ethyl-4-(4-methylphenyl)-6-neopentyl-1,4-dihydropyridine-3-carboxylate

A mixture of 5,5-dimethyl-3-oxohexanenitrile (3.5 g, 19 mmol), p-tolualdehyde (2.3 g, 19 mmol), piperidine (0.19 mL, 1.9 mmol), acetic acid (0.22 mL, 3.9 mmol) and toluene (200 mL) was heated under reflux for 3 hrs using a Dean-Stark trap. The reaction mixture was allowed to cool to room temperature, washed with saturated brine, dried over anhydrous magnesium sulfate and concentrated under reduced pressure to give 2-(3,3-dimethylbutanoyl)-3-(4-methylphenyl)acrylonitrile as a crude product (5.7 g). The crude product (5.7 g) and the crude product (2.5 g) obtained in Step A of Reference Example 4 were dissolved in acetic acid (15 mL), and the mixture was stirred at 80° C. for 30 min. The reaction mixture was concentrated under reduced pressure, and the residue was partitioned between ethyl acetate and saturated aqueous sodium hydrogencarbonate. The organic layer was washed with saturated brine, dried over anhydrous magnesium sulfate and concentrated under reduced pressure. The residue was purified by silica gel column chromatography and crystallized from hexane-ethyl acetate to give the title compound (3.1 g, yield 45%) as a white powder.

melting point: 126° C.

### Step C. methyl 5-cyano-2-ethyl-4-(4-methylphenyl)-6-neopentylnicotinate

To a solution of methyl 5-cyano-2-ethyl-4-(4-methylphenyl)-6-neopentyl-1,4-dihydropyridine-3-carboxylate (3.0 g, 8.6 mmol) in acetone (75 mL) was added dropwise a solution of cerium ammonium nitrate (11.8 g, 21 mmol) in water (15 mL) at room temperature. The obtained mixture was stirred at room temperature for 5 min and partitioned between ethyl acetate and water. The organic layer was washed successively with saturated aqueous sodium hydrogencarbonate and saturated brine, dried over anhydrous magnesium sulfate and concentrated under reduced pressure. The residue was crystallized from hexane-ethyl acetate to give the title compound (2.5 g, yield 83%) as a white powder.

MS 351(M+1).

### Step D. methyl 5-(aminomethyl)-2-ethyl-4-(4-methylphenyl)-6-neopentylnicotinate

A mixture of methyl 5-cyano-2-ethyl-4-(4-methylphenyl)-6-neopentylnicotinate (1.0 g, 2.9 mmol), Raney-nickel (5 mL), 25% aqueous ammonia (5 mL) and methanol (50 mL) was stirred at room temperature for 1 hr in a sealed tube under a hydrogen atmosphere at 0.3-0.5 MPa. The reaction mixture was filtered and the filtrate was concentrated under reduced pressure. The residue was purified by silica gel column chromatography to give the title compound (1.00 g, yield 99%) as a white powder.

MS 355(M+1).

### Step E. tert-butyl {[6-ethyl-5-(hydroxymethyl)-4-(4-methylphenyl)-2-neopentylpyridin-3-yl]methyl}carbamate

A solution of methyl 5-(aminomethyl)-2-ethyl-4-(4-methylphenyl)-6-neopentylnicotinate (0.50 g, 1.4 mmol) in toluene (10 mL) was cooled to −78° C., and 1 M diisobutylaluminum hydride toluene solution (3.3 mL, 4.9 mmol) was added dropwise over 30 min. The obtained mixture was allowed to warm and stirred at 0° C. for 15 min. To the reaction mixture was added isopropanol (2 mL) and then added tetrahydrofuran (10 mL) and saturated aqueous sodium hydrogencarbonate (4 mL) were added. The mixture was stirred at room temperature for 5 min. To the reaction mixture was added di-tert-butyl dicarbonate (0.49 mL, 2.1 mmol), and the mixture was stirred at room temperature for 1 hr. The reaction mixture was diluted with ethyl acetate, washed successively with 1N hydrochloric acid, saturated aqueous sodium hydrogencarbonate and saturated brine, dried over anhydrous magnesium sulfate and concentrated under reduced pressure. The residue was crystallized from hexane to give the title compound (0.48 g, yield 80%) as colorless crystals.

MS 427(M+1).

### Step F. tert-butyl {[5-(cyanomethyl)-6-ethyl-4-(4-methylphenyl)-2-neopentylpyridin-3-yl]methyl}carbamate

To an ice-cooled mixture of tert-butyl {[6-ethyl-5-(hydroxymethyl)-4-(4-methylphenyl)-2-neopentylpyridin-3-yl]methyl}carbamate (2.0 g, 4.8 mmol), triethylamine (1.3 mL, 9.6 mmol) and tetrahydrofuran (40 mL) was added dropwise methanesulfonyl chloride (0.56 mL, 7.2 mmol). The mixture was allowed to warm to room temperature and stirred for 30 min. The reaction mixture was diluted with ethyl acetate, washed successively with water, saturated aqueous sodium hydrogencarbonate and saturated brine, dried over anhydrous magnesium sulfate and concentrated under reduced pressure to give [5-{[(tert-butoxycarbonyl)amino]methyl}-2-ethyl-4-(4-methylphenyl)-6-neopentylpyridin-3-yl]methyl methanesulfonate as a crude product (2.6 g). The crude product (2.6 g) was dissolved in a mixture of acetonitrile (40 mL) and tetrahydrofuran (40 mL), trimethylsilane carbonitrile (0.77 mL, 5.7 mmol) and 1 M tetrabutylammonium fluoride tetrahydrofuran solution (5.7 mL, 5.7 mmol) were successively added thereto. The obtained mixture was stirred at room temperature for 10 min and concentrated under reduced pressure. The residue was partitioned between ethyl acetate and saturated brine, and the organic layer was dried over anhydrous magnesium sulfate and concentrated under reduced pressure. The residue was purified by silica gel column chromatography and crystallized from hexane-ethyl acetate to give the title compound (1.9 g, yield 92%) as a white powder.

MS 436(M+1).

### Step G. [5-{[(tert-butoxycarbonyl)amino]methyl}-2-ethyl-4-(4-methylphenyl)-6-neopentylpyridin-3-yl]acetic acid

tert-Butyl {[5-(cyanomethyl)-6-ethyl-4-(4-methylphenyl)-2-neopentylpyridin-3-yl]methyl}carbamate (1.9 g, 4.3 mmol) was suspended in 6N hydrochloric acid (100 mL), and the suspension was stirred at 90° C. for 24 hr. The reaction mixture was concentrated under reduced pressure, and the residue was partitioned between ethyl acetate and water. The aqueous layer was alkalified with saturated aqueous sodium hydrogencarbonate, and tetrahydrofuran (200 mL) and di-tert-butyl dicarbonate (1.5 mL, 6.5 mmol) were added. The mixture was stirred at room temperature for 17 hrs. The reaction mixture was acidified with 1N hydrochloric acid and partitioned. The aqueous layer was extracted with ethyl acetate. The organic layer and the extract were combined, and the mixture was washed with saturated brine, dried over anhydrous magnesium sulfate and concentrated under reduced pressure to give the title compound (1.8 g, yield 94%) as a white powder.

MS 455(M+1).

### Reference Example 5 1-{[5-(aminomethyl)-6-isobutyl-2-methyl-4-(4-methylphenyl)pyridin-3-yl]acetyl}-L-prolinamide dihydrochloride

### Step A. tert-butyl {[5-{2-[(2S)-2-(aminocarbonyl)pyrrolidin-1-yl]-2-oxoethyl}-2-isobutyl-6-methyl-4-(4-methylphenyl)pyridin-3-yl]methyl}carbamate

A mixture of [5-{[(tert-butoxycarbonyl)amino]methyl}-6-isobutyl-2-methyl-4-(4-methylphenyl)pyridin-3-yl]acetic acid (0.50 g, 1.2 mmol), L-prolinamide (0.32 g, 2.8 mmol), O-(7-azabenzotriazol-1-yl)-1,1,3,3-tetramethyluronium hexafluorophosphate (1.1 g, 2.8 mmol) and N,N-dimethylformamide (20 mL) was stirred at room temperature for 16 hrs. The reaction mixture was partitioned between ethyl acetate and water. The organic layer was washed successively with saturated aqueous sodium hydrogencarbonate and saturated brine, dried over anhydrous magnesium sulfate and concentrated under reduced pressure. The residue was purified by silica gel column chromatography to give the title compound (0.49 g, yield 81%) as a white powder.

MS 523(M+1).

### Step B. 1-{[5-(aminomethyl)-6-isobutyl-2-methyl-4-(4-methylphenyl)pyridin-3-yl]acetyl}-L-prolinamide dihydrochloride

A mixture of tert-butyl {[5-{2-[(2S)-2-(aminocarbonyl)pyrrolidin-1-yl]-2-oxoethyl}-2-isobutyl-6-methyl-4-(4-methylphenyl)pyridin-3-yl]methyl}carbamate (0.48 g, 0.90 mmol) and 4N hydrogen chloride 1,4-dioxane solution (5 mL) was stirred at room temperature for 2 hr. The reaction mixture was concentrated under reduced pressure and the residue was washed with diisopropyl ether to give the title compound (0.37 g, yield 82%) as a white powder.

MS 423(M+1).

### Reference Example 6 8-{[5-(aminomethyl)-6-isobutyl-2-methyl-4-(4-methylphenyl)pyridin-3-yl]acetyl}hexahydropyrazino[2,1-c][1,4]oxazin-4(3H)-one dihydrochloride

### Step A. 8-benzylhexahydropyrazino[2,1-c][1,4]oxazin-4(3H)-one

To a mixture of (4-benzylpiperazin-2-yl)methanol (6.4 g, 30 mmol) prepared by a method similar to the method described in J. Med. Chem. 1993, 36, 2075-2083, water (100 mL) and tetrahydrofuran (100 mL) were successively added potassium carbonate (8.3 g, 60 mmol) and chloroacetyl chloride (3.6 mL, 45 mmol), and the mixture was stirred at room temperature for 12 hr. The reaction mixture was concentrated under reduced pressure, and the residue was partitioned between ethyl acetate and water. The organic layer was washed with saturated brine, dried over anhydrous magnesium sulfate and concentrated under reduced pressure. The residue was dissolved in ethanol (100 mL), potassium hydroxide (2 g) was added, and the mixture was stirred at 50° C. for 3 hr. The reaction mixture was concentrated under reduced pressure, and the residue was partitioned between ethyl acetate and water. The organic layer was washed with saturated brine, dried over anhydrous magnesium sulfate and concentrated under reduced pressure to give the title compound (2.6 g, yield 35%) as a yellow oil.

MS 247(M+1).

### Step B. hexahydropyrazino[2,1-c][1,4]oxazin-4(3H)-one hydrochloride

To a solution of 8-benzylhexahydropyrazino[2,1-c][1,4]oxazin-4(3H)-one (2.6 g, 10.5 mmol) in methanol (50 mL) were added ammonium formate (3.0 g) and palladium-carbon (10%, 1.5 g), and the mixture was stirred at 80° C. for 15 min. The reaction mixture was allowed to cool to room temperature and filtrated to remove palladium-carbon. The filtrate was concentrated under reduced pressure, and 4N hydrogen chloride ethyl acetate solution was added to the residue. The precipitated crystals were collected by filtration, washed with ethyl acetate and dried under reduced pressure to give the title compound (1.8 g, yield 93%) as a pale-yellow powder.

MS 157(M+1).

### Step C. tert-butyl ({2-isobutyl-6-methyl-4-(4-methylphenyl)-5-[2-oxo-2-(4-oxohexahydropyrazino[2,1-c][1,4]oxazin-8(1H)-yl)ethyl]pyridin-3-yl}methyl)carbamate

[5-{[(tert-Butoxycarbonyl)amino]methyl}-6-isobutyl-2-methyl-4-(4-methylphenyl)pyridin-3-yl]acetic acid (0.43 g, 1.0 mmol) was dissolved in N,N-dimethylformamide (5 mL), and hexahydropyrazino[2,1-c][1,4]oxazin-4(3H)-one hydrochloride (0.29 g, 1.5 mmol), O-(7-azabenzotriazol-1-yl)-1,1,3,3-tetramethyluronium hexafluorophosphate (0.57 g, 1.5 mmol) and triethylamine (0.35 mL, 2.5 mmol) were added, and the mixture was stirred at room temperature for 12 hrs. The reaction mixture was partitioned between ethyl acetate and water. The organic layer was washed successively with 1N hydrochloric acid and saturated brine, dried over anhydrous magnesium sulfate and concentrated under reduced pressure. The residue was purified by silica gel column chromatography and crystallized from hexane-diethyl ether to give the title compound (0.42 g, yield 74%) as a white powder.

MS 565(M+1).

### Step D. 8-{[5-(aminomethyl)-6-isobutyl-2-methyl-4-(4-methylphenyl)pyridin-3-yl]acetyl}hexahydropyrazino[2,1-c][1,4]oxazin-4(3H)-one dihydrochloride

tert-Butyl({2-isobutyl-6-methyl-4-(4-methylphenyl)-5-[2-oxo-2-(4-oxohexahydropyrazino[2,1-c][1,4]oxazin-8(1H)-yl)ethyl]pyridin-3-yl}methyl)carbamate (0.42 g, 0.74 mmol) was dissolved in ethyl acetate (2 mL), 4N hydrogen chloride ethyl acetate solution (3 mL) was added, and the mixture was stirred at room temperature for 3 hrs. The reaction mixture was concentrated under reduced pressure and crystallized from hexane-diethyl ether to give the title compound (0.39 g, yield 98%) as a white powder.

MS 465(M+1).

### Reference Example 7 2-[5-(aminomethyl)-6-isobutyl-2-methyl-4-(4-methylphenyl)pyridin-3-yl]-N-[3-(methylsulfonyl)phenyl]acetamide dihydrochloride

### Step A. tert-butyl {[2-isobutyl-6-methyl-4-(4-methylphenyl)-5-(2-{[3-(methylsulfonyl)phenyl]amino}-2-oxoethyl)pyridin-3-yl]methyl}carbamate

The title compound (0.59 g, yield 87%) was obtained as crystals from [5-{[(tert-butoxycarbonyl)amino]methyl}-6-isobutyl-2-methyl-4-(4-methylphenyl)pyridin-3-yl]acetic acid (0.50 g, 1.17 mmol) and 3-methylsulfonylaniline hydrochloride (0.24 g, 1.17 mmol) by a method similar to Step C of Reference Example 6.

¹H-NMR (CDCl₃) δ: 0.98 (6H, d, J=6.6 Hz), 1.38 (9H, s), 2.18-2.27 (1H, m), 2.41 (3H, s), 2.63 (3H, s), 2.77 (2H, d, J=7.4 Hz), 3.05 (3H, s), 3.49 (2H, s), 4.06 (2H, d, J=5.1 Hz), 4.23 (1H, s), 6.90 (1H, m), 7.01 (2H, d, J=7.9 Hz), 7.25 (2H, d, J=7.9 Hz), 7.50 (1H, t, J=7.9 Hz), 7.64-7.67 (1H, m), 7.76 (1H, d, J=8.7 Hz), 7.83 (1H, t, J=1.9 Hz).

### Step B. 2-[5-(aminomethyl)-6-isobutyl-2-methyl-4-(4-methylphenyl)pyridin-3-yl]-N-[3-(methylsulfonyl)phenyl]acetamide dihydrochloride

To a solution of tert-butyl {[2-isobutyl-6-methyl-4-(4-methylphenyl)-5-(2-{[3-(methylsulfonyl)phenyl]amino}-2-oxoethyl)pyridin-3-yl]methyl}carbamate (0.45 g, 0.78 mmol) in tetrahydrofuran (4 mL) was added 4N hydrogen chloride ethyl acetate solution (10 mL), and the mixture was stirred at room temperature for 16 hrs. The precipitated crystals were collected by filtration, washed with ethyl acetate and recrystallized from methanol-ethyl acetate to give the title compound (0.31 g, yield 56%) as crystals.

¹H-NMR (DMSO-d₆) δ: 0.99 (6H, d, J=6.6 Hz), 2.14-2.23 (1H, m), 2.36 (3H, s), 2.80 (3H, s), 3.12-3.18 (2H, m), 3.18 (3H, s), 3.63 (2H, s), 3.79-3.84 (2H, m), 7.21 (2H, d, J=8.0 Hz), 7.34 (2H, d, J=8.0 Hz), 7.55-7.64 (2H, m), 7.70-7.74 (1H, m), 8.16-8.18 (1H, m), 8.37 (3H, s), 10.6 (1H, s).

TABLE 2

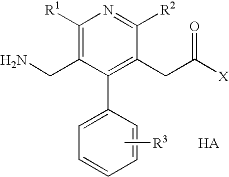

| Ex. | R¹ | R² | R³ | —X | HA |
|---|---|---|---|---|---|
| 1 | isobutyl | ethyl | 4-methyl | —OH | none |
| 2 | isobutyl | isobutyl | 4-methyl | —OH | 2HCl |
| 3 | neopentyl | ethyl | 4-methyl | —OH | 2HCl |
| 4 | neopentyl | ethyl | 4-methyl | —OH | none |
| 5 | neopentyl | ethyl | 4-methyl | 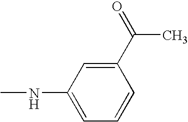 | 2HCl |
| 6 | neopentyl | ethyl | 4-methyl | 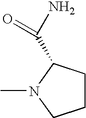 | none |
| 7 | neopentyl | ethyl | 4-methyl | 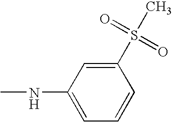 | 2HCl |
| 8 | neopentyl | ethyl | 4-methyl | —O—CH₃ | 2HCl |
| 9 | neopentyl | ethyl | 4-methyl | 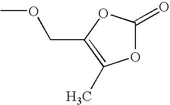 | 2HCl |
| 10 | isobutyl | isobutyl | 4-methyl | 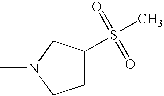 | 2CF₃COOH |

### Example 1 [5-(aminomethyl)-2-ethyl-6-isobutyl-4-(4-methylphenyl)pyridin-3-yl]acetic acid

### Step A. methyl 5-cyano-2-ethyl-6-isobutyl-4-(4-methylphenyl)-1,4-dihydropyridine-3-carboxylate

Methyl 3-aminopent-2-enoate was obtained as a crude product (11.5 g) from methyl 3-oxopentanoate (12.0 g, 92 mmol) by a method similar to Step A of Reference Example 4. A mixture of 5-methyl-3-oxohexanenitrile (11.4 g, 91 mmol), p-tolualdehyde (11.0 g, 91 mmol), piperidine (0.90 mL, 9.1 mmol), acetic acid (1.05 mL, 18 mmol) and toluene (200 mL) was heated under reflux for 12 hrs using a Dean-Stark trap. The reaction mixture was allowed to cool to room temperature, washed with saturated brine, dried over anhydrous magnesium sulfate and concentrated under reduced pressure to give 2-(3-methylbutanoyl)-3-(4-methylphenyl)acrylonitrile as a crude product (21 g). The crude product (21 g) and the aforementioned crude product (11.5 g) of methyl 3-aminopent-2-enoate were dissolved in acetic acid (20 mL)), and the mixture was stirred at 90° C. for 2 hr. The reaction mixture was concentrated under reduced pressure and the obtained orange oil was washed with hexane to give the title compound (29.7 g, yield 96%) as an orange oil.

MS 339(M+1).

### Step B. methyl 5-cyano-2-ethyl-6-isobutyl-4-(4-methylphenyl)nicotinate

To a solution of methyl 5-cyano-2-ethyl-6-isobutyl-4-(4-methylphenyl)-1,4-dihydropyridine-3-carboxylate (29.7 g, 88 mmol) in acetone (750 mL) was added dropwise a solution of cerium ammonium nitrate (120 g, 0.21 mol) in water (150 mL). The obtained mixture was stirred at room temperature for 5 min. The reaction mixture was partitioned between ethyl acetate and water, and the organic layer was washed successively with saturated aqueous sodium hydrogencarbonate and saturated brine, dried over anhydrous magnesium sulfate and concentrated under reduced pressure. The residue was purified by silica gel column chromatography and crystallized from hexane to give the title compound (13.9 g, yield 47%) as a white powder.

MS 337(M+1).

### Step C. methyl 5-(aminomethyl)-2-ethyl-6-isobutyl-4-(4-methylphenyl)nicotinate

A mixture of methyl 5-cyano-2-ethyl-6-isobutyl-4-(4-methylphenyl)nicotinate (22.7 g, 68 mmol), Raney-nickel (25 mL), 25% aqueous ammonia (25 mL) and methanol (200 mL) was stirred at 50° C. for 4 hr in a sealed tube under a hydrogen atmosphere at 0.3-0.5 MPa. The reaction mixture was filtrated and the filtrate was concentrated under reduced pressure. The residue was dissolved in 1N hydrochloric acid and the solution was washed with ethyl acetate. The aqueous layer was separated, alkalified with 5% aqueous ammonia and extracted with ethyl acetate. The extract was washed with saturated brine, dried over anhydrous magnesium sulfate and concentrated under reduced pressure to give the title compound (20.7 g, yield 90%) as a pale-orange oil.

MS 341(M+1).

### Step D. tert-butyl {[6-ethyl-5-(hydroxymethyl)-2-isobutyl-4-(4-methylphenyl)pyridin-3-yl]methyl}carbamate

A solution of methyl 5-(aminomethyl)-2-ethyl-6-isobutyl-4-(4-methylphenyl)nicotinate (20.6 g, 61 mmol) in toluene (280 mL) was cooled to −78° C., 1 M diisobutylaluminum hydride toluene solution (141 mL, 0.21 mol) was added dropwise over 90 min. The obtained mixture was stirred at the same temperature for 10 min, and ethyl acetate (20 mL) and sodium sulfate decahydrate (69 g) were successively added. The reaction mixture was allowed to warm to room temperature and stirred for 12 hr. Insoluble materials were filtered off and washed with toluene. The filtrate and the washing solution were combined, and the mixture was concentrated under reduced pressure. The residue was dissolved in tetrahydrofuran (180 mL), di-tert-butyl dicarbonate (14.5 mL, 63 mmol) was added, and the mixture was stirred at room temperature for 1 hr. The reaction mixture was concentrated under reduced pressure, and the residue was crystallized from hexane-diisopropyl ether to give the title compound (15.7 g, yield 63%) as a gray-white powder.

MS 413(M+1).

### Step E. tert-butyl {[5-(cyanomethyl)-6-ethyl-2-isobutyl-4-(4-methylphenyl)pyridin-3-yl]methyl}carbamate

To an ice-cooled mixture of tert-butyl {[6-ethyl-5-(hydroxymethyl)-2-isobutyl-4-(4-methylphenyl)pyridin-3-yl]methyl}carbamate (15.7 g, 38 mmol), triethylamine (10.6 mL, 76 mmol) and tetrahydrofuran (150 mL) was added dropwise methanesulfonyl chloride (4.4 mL, 57 mmol), and the mixture was stirred at 5° C. for 30 min. Water was added to the reaction mixture, and the mixture was extracted with ethyl acetate. The extract was washed successively with saturated aqueous sodium hydrogencarbonate and saturated brine, dried over anhydrous magnesium sulfate and concentrated under reduced pressure to give [5-{[(tert-butoxycarbonyl)amino]methyl}-2-ethyl-6-isobutyl-4-(4-methylphenyl)pyridin-3-yl]methyl methanesulfonate as a crude product (23 g). The crude product (23 g) was dissolved in a mixture of acetonitrile (150 mL) and tetrahydrofuran (150 mL), trimethylsilane carbonitrile (6.1 mL, 46 mmol) and 1 M tetrabutylammonium fluoride tetrahydrofuran solution (46 mL, 46 mmol) were successively added thereto. The obtained mixture was stirred at room temperature for 30 min., and concentrated under reduced pressure. The residue was partitioned between ethyl acetate and water, and the organic layer was washed successively with saturated aqueous sodium hydrogencarbonate and saturated brine, dried over anhydrous magnesium sulfate and concentrated under reduced pressure. The residue was purified by silica gel column chromatography to give the title compound (15.7 g, yield 98%) as a white powder.

MS 422(M+1).

### Step F. [5-(aminomethyl)-2-ethyl-6-isobutyl-4-(4-methylphenyl)pyridin-3-yl]acetic acid

tert-Butyl {[5-(cyanomethyl)-6-ethyl-2-isobutyl-4-(4-methylphenyl)pyridin-3-yl]methyl}carbamate (15.6 g, 37 mmol) was suspended in 6N hydrochloric acid (60 mL), and the suspension was stirred at 90° C. for 24 hrs. The reaction mixture was concentrated under reduced pressure. The residue was dissolved in water (150 mL), and the solution was washed with ethyl acetate. The aqueous layer was stirred at 5° C., and neutralized with 8N aqueous sodium hydroxide solution. The obtained suspension was stirred at the same temperature for 2 hrs, and the precipitate was collected by filtration, washed with water and dried to give monohydrate of the title compound (9.1 g, yield 72%) as a pale-yellow powder.

Elemental analysis for C₂₁H₂₈N₂O₂H₂O

Calculated: C, 70.36; H, 8.44; N, 7.81.

Found: C, 69.95; H, 8.18; N, 7.54.

MS 341(M+1).

### Example 2 [5-(aminomethyl)-2,6-diisobutyl-4-(4-methylphenyl)pyridin-3-yl]acetic acid dihydrochloride

A mixture of [5-{[(tert-butoxycarbonyl)amino]methyl}-2,6-diisobutyl-4-(4-methylphenyl)pyridin-3-yl]acetic acid (0.050 g, 0.11 mmol) prepared in Reference Example 3 and 4N hydrogen chloride 1,4-dioxane solution (5 mL) was stirred at room temperature for 2 hr. The reaction mixture was concentrated under reduced pressure, and the residue was triturated from diisopropyl ether to give the title compound (0.048 g, yield 100%) as a pale-yellow powder.

MS 369(M+1).

### Example 3 [5-(aminomethyl)-2-ethyl-4-(4-methylphenyl)-6-neopentylpyridin-3-yl]acetic acid dihydrochloride

[5-{[(tert-Butoxycarbonyl)amino]methyl}-2-ethyl-4-(4-methylphenyl)-6-neopentylpyridin-3-yl]acetic acid (0.14 g, 0.31 mmol) prepared in Reference Example 4 was suspended in 6N hydrochloric acid (5 mL), and the suspension was stirred at room temperature for 3 hr. The reaction mixture was concentrated under reduced pressure, and the residue was triturated from diisopropyl ether to give the title compound (0.13 g, yield 99%) as a white powder.

MS 355(M+1).

### Example 4 [5-(aminomethyl)-2-ethyl-4-(4-methylphenyl)-6-neopentylpyridin-3-yl]acetic acid

[5-(Aminomethyl)-2-ethyl-4-(4-methylphenyl)-6-neopentylpyridin-3-yl]acetic acid dihydrochloride (1.0 g, 2.3 mmol) was dissolved in water (2 mL), and the solution was neutralized with 4N aqueous sodium hydroxide solution under ice-cooling. The obtained suspension was stirred at 5° C. for 1 hr and the resulting crystals were collected by filtration. The obtained crystals were washed with cold water and dried to give the title compound (0.69 g, yield 83%) as a white powder.

MS 355(M+1).

[5-(Aminomethyl)-2-ethyl-4-(4-methylphenyl)-6-neopentylpyridin-3-yl]acetic acid dihydrochloride (3.6 g, 8.4 mmol) was dissolved in water (7.2 mL), and the solution was neutralized with 4N aqueous sodium hydroxide solution under ice-cooling. The obtained suspension was stirred at 5° C. for 1 hr and the resulting crystals were collected by filtration. The obtained crystals were washed three times with cold water (5 ml) and dried to give monohydrate of the title compound (2.4 g, yield 80%) as a white powder.

Elemental analysis for C₂₂H₃₀N₂O₂H₂O

Calculated: C, 70.94; H, 8.66; N, 7.52.

Found: C, 71.12; H, 8.52; N, 7.45.

### Example 5N-(3-acetylphenyl)-2-[5-(aminomethyl)-2-ethyl-4-(4-methylphenyl)-6-neopentylpyridin-3-yl]acetamide dihydrochloride

### Step A. tert-butyl {[5-{2-[(3-acetylphenyl)amino]-2-oxoethyl}-6-ethyl-4-(4-methylphenyl)-2-neopentylpyridin-3-yl]methyl}carbamate

To a solution of [5-{[(tert-butoxycarbonyl)amino]methyl}-2-ethyl-4-(4-methylphenyl)-6-neopentylpyridin-3-yl]acetic acid (0.35 g, 0.77 mmol) prepared in Reference Example 4 in N,N-dimethylformamide (5 mL) were successively added 3-aminoacetophenone (0.16 g, 1.2 mmol), N,N-diisopropylethylamine (0.20 mL, 1.2 mmol) and O-(7-azabenzotriazol-1-yl)-1,1,3,3-tetramethyluronium hexafluorophosphate (0.44 g, 1.2 mmol). The mixture was stirred at room temperature for 17 hrs., and partitioned between ethyl acetate and water. The organic layer was washed successively with saturated aqueous sodium hydrogencarbonate and saturated brine, dried over anhydrous magnesium sulfate and concentrated under reduced pressure. The residue was purified by silica gel column chromatography and crystallized from diisopropyl ether-ethyl acetate to give the title compound (0.36 g, yield 82%) as a white powder.

MS 572(M+1).

### Step B. N-(3-acetylphenyl)-2-[5-(aminomethyl)-2-ethyl-4-(4-methylphenyl)-6-neopentylpyridin-3-yl]acetamide dihydrochloride

tert-Butyl {[5-{2-[(3-acetylphenyl)amino]-2-oxoethyl}-6-ethyl-4-(4-methylphenyl)-2-neopentylpyridin-3-yl]methyl}carbamate (0.32 g, 0.56 mmol) was dissolved in 4N hydrogen chloride ethyl acetate solution (5 mL), and the solution was stirred at room temperature for 1 hr. The reaction mixture was concentrated under reduced pressure, and the residue was crystallized from diisopropyl ether-methanol to give the title compound (0.28 g, yield 90%) as a white powder.

MS 472(M+1).

### Example 6 1-{[5-(aminomethyl)-2-ethyl-4-(4-methylphenyl)-6-neopentylpyridin-3-yl]acetyl}-L-prolinamide

### Step A. tert-butyl {[5-{2-[(2S)-2-(aminocarbonyl)pyrrolidin-1-yl]-2-oxoethyl}-6-ethyl-4-(4-methylphenyl)-2-neopentylpyridin-3-yl]methyl}carbamate

The title compound (0.14 g, yield 98%) was obtained as a white powder from [5-{[(tert-butoxycarbonyl)amino]methyl}-2-ethyl-4-(4-methylphenyl)-6-neopentylpyridin-3-yl]acetic acid (0.12 g, 0.26 mmol) prepared in Reference Example 4 and L-prolinamide (0.046 g, 0.4 mmol) by a method similar to Step A of Example 5.

MS 551(M+1).

### Step B. 1-{[5-(aminomethyl)-2-ethyl-4-(4-methylphenyl)-6-neopentylpyridin-3-yl]acetyl}-L-prolinamide

To a solution of tert-butyl {[5-{2-[(2S)-2-(aminocarbonyl)pyrrolidin-1-yl]-2-oxoethyl}-6-ethyl-4-(4-methylphenyl)-2-neopentylpyridin-3-yl]methyl}carbamate (0.14 g, 0.26 mmol) in ethyl acetate (2 mL) was added 4N hydrogen chloride ethyl acetate solution (3 mL), and the mixture was stirred at room temperature for 3 hr. Water was added to the reaction mixture, and the mixture was washed with ethyl acetate. The aqueous layer was alkalified (pH 8.0) with 25% aqueous ammonia solution and extracted with ethyl acetate. The extract was washed with saturated brine, dried over anhydrous magnesium sulfate and concentrated under reduced pressure. The residue was purified by silica gel column chromatography and crystallized from hexane-diethyl ether to give monohydrate of the title compound (0.057 g, yield 42%) as a white powder.

Elemental analysis for C₂₇H₃₈N₄O₂H₂O

Calculated: C, 69.20; H, 8.60; N, 11.96.

Found: C, 69.23; H, 8.54; N, 11.53.

MS 451(M+1).

### Example 72-[5-(aminomethyl)-2-ethyl-4-(4-methylphenyl)-6-neopentylpyridin-3-yl]-N-[3-(methylsulfonyl)phenyl]acetamide dihydrochloride

### Step A. tert-butyl{[6-ethyl-4-(4-methylphenyl)-5-(2-{[3-(methylsulfonyl)phenyl]amino}-2-oxoethyl)-2-neopentylpyridin-3-yl]methyl}carbamate

To a solution of [5-{[(tert-butoxycarbonyl)amino]methyl}-2-ethyl-4-(4-methylphenyl)-6-neopentylpyridin-3-yl]acetic acid (0.18 g, 0.40 mmol) prepared in Reference Example 4 in N,N-dimethylformamide (5 mL) were successively added 3-methylsulfonylaniline hydrochloride (0.12 g, 0.59 mmol), N,N-diisopropylethylamine (0.21 mL, 1.2 mmol) and O-(7-azabenzotriazol-1-yl)-1,1,3,3-tetramethyluronium hexafluorophosphate (0.23 g, 0.59 mmol). The mixture was stirred at room temperature for 17 hrs., and partitioned between ethyl acetate and water. The organic layer was washed successively with saturated aqueous sodium hydrogencarbonate and saturated brine, dried over anhydrous magnesium sulfate and concentrated under reduced pressure. The residue was purified by silica gel column chromatography and crystallized from diisopropyl ether-ethyl acetate to give the title compound (0.19 g, yield 77%) as a pale-yellow powder.

MS 608(M+1).

### Step B. 2-[5-(aminomethyl)-2-ethyl-4-(4-methylphenyl)-6-neopentylpyridin-3-yl]-N-[3-(methylsulfonyl)phenyl]acetamide dihydrochloride

The title compound (0.12 g, yield 85%) was obtained as pale-yellow crystals from tert-butyl {[6-ethyl-4-(4-methylphenyl)-5-(2-{[3-(methylsulfonyl)phenyl]amino}-2-oxoethyl)-2-neopentylpyridin-3-yl]methyl}carbamate (0.15 g, 0.25 mmol) by a method similar to Step B of Example 5.

MS 508(M+1).

### Example 8 methyl [5-(aminomethyl)-2-ethyl-4-(4-methylphenyl)-6-neopentylpyridin-3-yl]acetate dihydrochloride

A mixture of [5-{[(tert-butoxycarbonyl)amino]methyl}-2-ethyl-4-(4-methylphenyl)-6-neopentylpyridin-3-yl]acetic acid (0.20 g, 0.44 mmol) and 10% hydrogen chloride methanol solution (3 mL) was stirred under heating at 80° C. for 3 hrs. The reaction mixture was concentrated and partitioned between ethyl acetate and 1N aqueous sodium hydroxide solution. The organic layer was washed with saturated brine, dried over anhydrous magnesium sulfate and concentrated under reduced pressure. The residue was purified by silica gel column chromatography. To the obtained colorless oil was added 4N hydrogen chloride ethyl acetate solution (1 mL) and the mixture was stirred. The precipitated crystals were collected by filtration and washed with ethyl acetate to give the title compound (0.18 g, yield 91%) as a white powder.

MS 369(M+1).

### Example 9 (5-methyl-2-oxo-1,3-dioxol-4-yl)methyl [5-(aminomethyl)-2-ethyl-4-(4-methylphenyl)-6-neopentylpyridin-3-yl]acetate dihydrochloride

### Step A. (5-methyl-2-oxo-1,3-dioxol-4-yl)methyl [5-{[(tert-butoxycarbonyl)amino]methyl}-2-ethyl-4-(4-methylphenyl)-6-neopentylpyridin-3-yl]acetate

A mixture of [5-{[(tert-butoxycarbonyl)amino]methyl}-2-ethyl-4-(4-methylphenyl)-6-neopentylpyridin-3-yl]acetic acid (0.77 g, 1.7 mmol), 4-(chloromethyl)-5-methyl-1,3-dioxol-2-one (0.38 g, 2.5 mmol), potassium carbonate (0.35 g, 2.5 mmol) and N,N-dimethylformamide (10 mL) was stirred at 60° C. for 2 hr. The reaction mixture was partitioned between ethyl acetate and water. The organic layer was washed successively with water and saturated brine, dried over anhydrous magnesium sulfate and concentrated under reduced pressure. The residue was purified by silica gel column chromatography and crystallized from diisopropyl ether to give the title compound (0.67 g, yield 71%) as a white powder.

MS 567(M+1).

### Step B. (5-methyl-2-oxo-1,3-dioxol-4-yl)methyl [5-(aminomethyl)-2-ethyl-4-(4-methylphenyl)-6-neopentylpyridin-3-yl]acetate dihydrochloride

The title compound (0.60 g, yield 99%) was obtained as a white powder from (5-methyl-2-oxo-1,3-dioxol-4-yl)methyl [5-{[(tert-butoxycarbonyl)amino]methyl}-2-ethyl-4-(4-methylphenyl)-6-neopentylpyridin-3-yl]acetate (0.63 g, 1.1 mmol) by a method similar to Example 2.

MS 467(M+1).

### Example 10 [(2,6-diisobutyl-4-(4-methylphenyl)-5-{2-[3-(methylsulfonyl)pyrrolidin-1-yl]-2-oxoethyl}pyridin-3-yl)methyl]amine ditrifluoroacetate

To 0.12 M solution (0.5 mL) of [5-{[(tert-butoxycarbonyl)amino]methyl}-2,6-diisobutyl-4-(4-methylphenyl)pyridin-3-yl]acetic acid (0.060 mmol) in N,N-dimethylformamide were successively added 0.24 M solution (0.5 mL) of 3-(methylsulfonyl)pyrrolidine (0.12 mmol) in N,N-dimethylformamide and 0.24 M solution (0.5 mL) of O-(7-azabenzotriazol-1-yl)-1,1,3,3-tetramethyluronium hexafluorophosphate (0.12 mmol) in N,N-dimethylformamide, and the mixture was stirred at room temperature for 17 hrs. The reaction mixture was diluted with dichloromethane (2 mL), and washed successively with saturated aqueous sodium hydrogencarbonate and water. The organic layer was separated, trifluoroacetic acid (1 mL) was added and the mixture was stirred at room temperature for 1 hr. The reaction mixture was concentrated under reduced pressure, and the residue was purified by HPLC to give the title compound (0.035 g, yield 81%).

MS 500(M+1).

The compounds of Examples 11 to 53 were prepared by a method similar to Example 10 from [5-{[(tert-butoxycarbonyl)amino]methyl}-2,6-diisobutyl-4-(4-methylphenyl)pyridin-3-yl]acetic acid and an amine corresponding to Table 3 or a free amine prepared from a salt of the amine.

TABLE 31

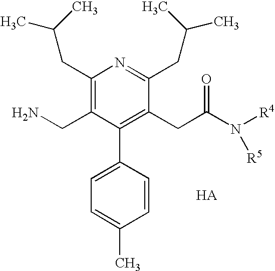

| | —NR⁴R⁵ | MS (M + 1) | HA |
|---|---|---|---|
| Ex. | | | |
| 11 | 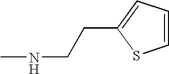 | 478 | 2CF₃COOH |
| 12 | 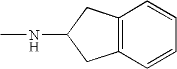 | 484 | 2CF₃COOH |
| 13 | 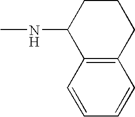 | 498 | 2CF₃COOH |
| 14 | 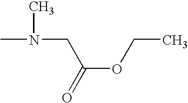 | 468 | 2CF₃COOH |
| 15 | 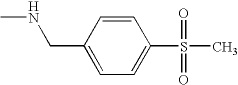 | 536 | 2CF₃COOH |
| 16 | 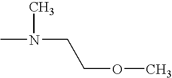 | 440 | 2CF₃COOH |
| 17 | 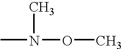 | 412 | 2CF₃COOH |
| 18 | 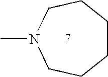 | 450 | 2CF₃COOH |
| 19 | 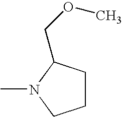 | 466 | 2CF₃COOH |
| 20 | 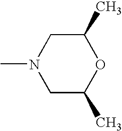 | 466 | 2CF₃COOH |
| 21 | 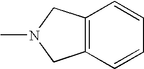 | 470 | 2CF₃COOH |
| 22 | 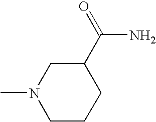 | 479 | 2CF₃COOH |
| 23 | 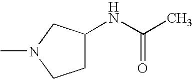 | 479 | 2CF₃COOH |
| 24 | 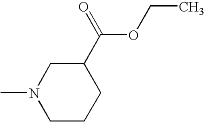 | 508 | 2CF₃COOH |
| 25 | 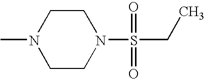 | 529 | 2CF₃COOH |
| 26 | 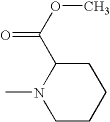 | 494 | 2CF₃COOH |
| 27 | 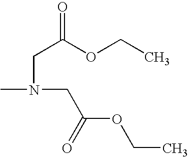 | 540 | 2CF₃COOH |
| 28 | 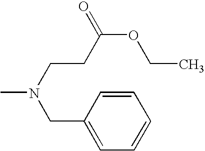 | 558 | 2CF₃COOH |
| 29 | 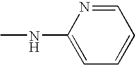 | 445 | 3CF₃COOH |
| 30 | 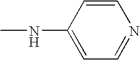 | 445 | 3CF₃COOH |
| 31 | 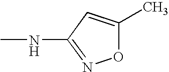 | 449 | 2CF₃COOH |
| 32 | 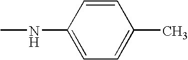 | 458 | 2CF₃COOH |
| 33 | 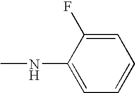 | 462 | 2CF₃COOH |
| 34 | 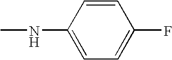 | 462 | 2CF₃COOH |
| 35 | 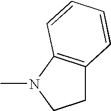 | 470 | 2CF₃COOH |
| 36 | 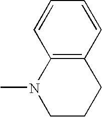 | 484 | 2CF₃COOH |
| 37 | 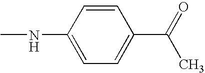 | 486 | 2CF₃COOH |
| Example | | | |
| 38 | 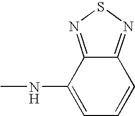 | 502 | 2CF₃COOH |
| 39 | 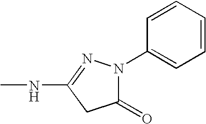 | 526 | 2CF₃COOH |
| 40 | 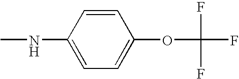 | 528 | 2CF₃COOH |
| 41 | 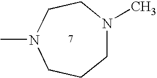 | 465 | 3CF₃COOH |
| 42 | 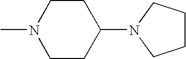 | 505 | 3CF₃COOH |
| 43 | 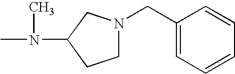 | 541 | 3CF₃COOH |
| 44 | 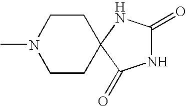 | 520 | 2CF₃COOH |
| 45 | 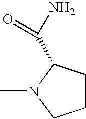 | 465 | 2CF₃COOH |
| 46 | 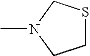 | 440 | 2CF₃COOH |
| 47 | 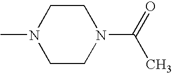 | 479 | 2CF₃COOH |
| 48 | 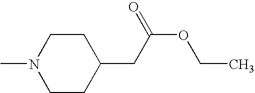 | 522 | 2CF₃COOH |
| 49 | 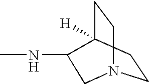 | 477 | 3CF₃COOH |
| 50 | 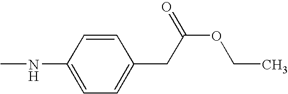 | 530 | 2CF₃COOH |
| 51 | 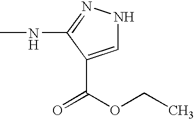 | 506 | 2CF₃COOH |
| 52 | 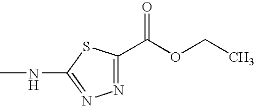 | 524 | 2CF₃COOH |
| 53 | 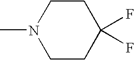 | 472 | 2CF₃COOH |

The compounds of Examples 54-94 were prepared by a method similar to Example 10 from [5-{[(tert-butoxycarbonyl)amino]methyl}-2-ethyl-4-(4-methylphenyl)-6-neopentylpyridin-3-yl]acetic acid and an amine corresponding to Table 4 or a free amine prepared from a salt of the amine.

TABLE 4

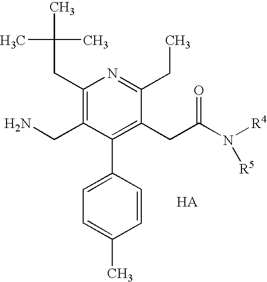

| | —NR⁴R⁵ | MS (M + 1) | HA |
|---|---|---|---|
| Ex. | | | |
| 54 | 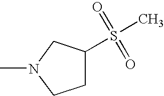 | 486 | 2CF₃COOH |
| 55 | 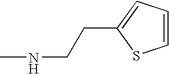 | 464 | 2CF₃COOH |
| 56 | 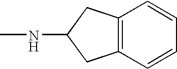 | 470 | 2CF₃COOH |
| 57 | 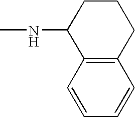 | 484 | 2CF₃COOH |
| 58 | 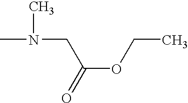 | 454 | 2CF₃COOH |
| 59 | 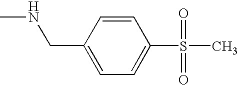 | 522 | 2CF₃COOH |
| 60 | 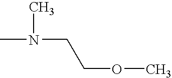 | 426 | 2CF₃COOH |
| 61 | 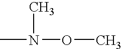 | 398 | 2CF₃COOH |
| 62 | 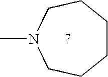 | 436 | 2CF₃COOH |
| 63 | 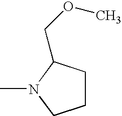 | 452 | 2CF₃COOH |
| 64 | 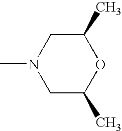 | 452 | 2CF₃COOH |
| 65 | 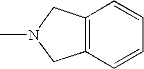 | 456 | 2CF₃COOH |
| 66 | 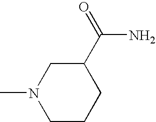 | 465 | 2CF₃COOH |
| 67 | 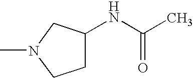 | 465 | 2CF₃COOH |
| 68 | 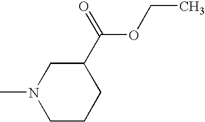 | 494 | 2CF₃COOH |
| 69 | 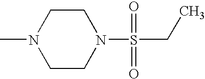 | 515 | 2CF₃COOH |
| 70 | 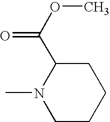 | 480 | 2CF₃COOH |
| 71 | 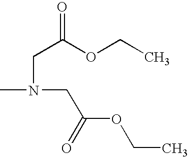 | 526 | 2CF₃COOH |
| 72 | 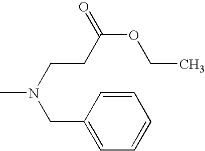 | 544 | 2CF₃COOH |
| 73 | 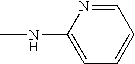 | 431 | 3CF₃COOH |
| 74 | 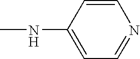 | 431 | 3CF₃COOH |
| 75 | 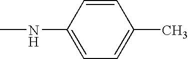 | 444 | 2CF₃COOH |
| 76 | 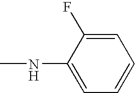 | 448 | 2CF₃COOH |
| 77 | 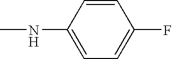 | 448 | 2CF₃COOH |
| 78 | 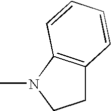 | 456 | 2CF₃COOH |
| 79 | 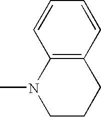 | 470 | 2CF₃COOH |
| 80 | 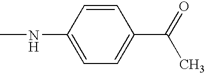 | 472 | 2CF₃COOH |
| Example | | | |
| 81 | 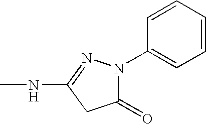 | 512 | 2CF₃COOH |
| 82 | 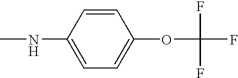 | 514 | 2CF₃COOH |
| 83 | 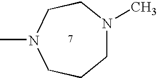 | 451 | 3CF₃COOH |
| 84 | 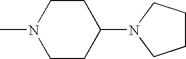 | 491 | 3CF₃COOH |
| 85 | 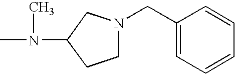 | 527 | 3CF₃COOH |
| 86 | 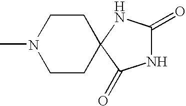 | 506 | 2CF₃COOH |
| 87 | 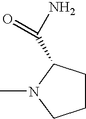 | 451 | 2CF₃COOH |
| 88 | 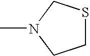 | 426 | 2CF₃COOH |
| 89 | 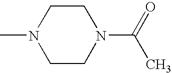 | 465 | 2CF₃COOH |
| 90 | 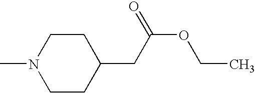 | 508 | 2CF₃COOH |
| 91 | 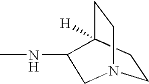 | 463 | 3CF₃COOH |
| 92 | 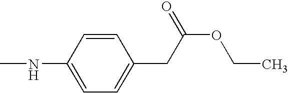 | 516 | 2CF₃COOH |
| 93 | 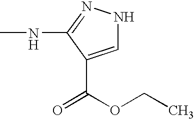 | 492 | 2CF₃COOH |
| 94 | 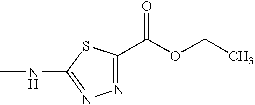 | 510 | 2CF₃COOH |

### Experimental Example 1

### Determination of Dipeptidyl Peptidase IV Inhibitory Activity in Rat Plasma

The reaction was carried out according to the method of Raymond et al. (Diabetes, vol. 47, pp. 1253-1258, 1998) using a 96 well flat-bottomed plate at 30° C. A dimethylformamide solution (1 μL) containing the test compound was added to a mixture of water (69 μL), 1 M Tris-hydrochloride buffer (10 μL, pH 7.5) and 1 mM aqueous Gly-Pro-p-NA solution (100 μL) to prepare a mixed solution. Plasma (20 μL) prepared from blood of SD rat according to a conventional method was added to the above-mentioned mixed solution and the enzyme reaction was started at 30° C. The absorbance after 0 hr. and 1 hr. was measured using a microplate reader at a wavelength of 405 nm and an increase (AODs) was determined. At the same time, an increase (AODc) in absorbance of the reaction mixture without the test compound, and an increase (AODb) in absorbance of the reaction mixture without the test compound and the enzyme were determined and the inhibition rate of dipeptidyl peptidase IV enzyme activity was calculated from the following formula:

{1−[(ΔODs−ΔODb)/(ΔODc−ΔODb)]}×100

The dipeptidyl peptidase IV inhibitory activity of the test compound group is expressed in IC₅₀ value (nM) and shown in Table 5.

TABLE 5

| Test compound | IC₅₀ value |
|---|---|
| (Example No.) | (nM) |
| 1 | 33 |
| 4 | 78 |
| 10 | 0.85 |
| 20 | 8.4 |
| 45 | 2.0 |
| 47 | 4.8 |
| 64 | 1.7 |
| 80 | 11 |
| 87 | 0.32 |
| 89 | 3.6 |

### Experimental Example 2

### Determination of Dipeptidyl Peptidase IV Inhibitory Activity in Rat Plasma

In the same manner as in Experimental Example 1, the inhibition rate of the dipeptidylpeptidase IV enzyme activity was determined using a solution (1 μL) of the test compound in N,N-dimethylformamide.

The dipeptidyl peptidase IV inhibitory activity of the test compound group is expressed in IC₅₀ value (nM) and shown in Table 6.

TABLE 6

| Test compound | IC₅₀ value |
|---|---|
| (Example No.) | (nM) |
| 2 | 35 |
| 3 | 35 |
| 5 | 5.1 |
| 6 | 2.7 |
| 7 | 51 |

As shown above, the compound of the present invention has a superior dipeptidyl peptidase IV inhibitory activity, and is useful as an agent for the prophylaxis or treatment of diabetes and the like.

### Formulation Example 1

### Production of Capsules

| | |
|---|---|
| 1) compound of Example 1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 2

### Production of Tablets

| | | |
|---|---|---|
| 1) compound of Example 1 | 30 | g |
| 2) lactose | 50 | g |
| 3) corn starch | 15 | g |
| 4) carboxymethylcellulose calcium | 44 | g |
| 5) magnesium stearate | 1 | g |
| total of 1000 tablets | 140 | g |

The entire amounts of 1), 2) and 3), and 30 g of 4) are kneaded with water, dried in vacuo and granulated. The granules are mixed with 14 g of 4) and 1 g of 5) and the mixture is compressed with a tableting machine, whereby 1000 tablets containing 30 mg of compound of Example 1 per tablet are obtained.

### INDUSTRIAL APPLICABILITY

The compound of the present invention shows a superior peptidase-inhibitory activity and is useful as an agent for the prophylaxis or treatment of diabetes and the like.

This application is based on patent application No. 52018/2005 filed in Japan, the contents of which are hereby incorporated by reference.

1. A compound represented by the formula (I): 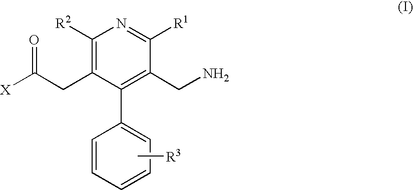
wherein
R¹ is a C₁₋₆ alkyl group optionally substituted by a C₃₋₁₀ cycloalkyl group,
R² is a C₂₋₆ alkyl group,
R³ is a hydrogen atom, a C₁₋₆ alkyl group or a halogen atom, and
X is —OR⁶or —NR⁴R⁵
wherein R⁴ and R⁶ are each independently a hydrogen atom, an optionally substituted hydrocarbon group or an optionally substituted heterocyclic group, R⁵ is an optionally substituted hydrocarbon group, an optionally substituted heterocyclic group or an optionally substituted hydroxy group, or R⁴ and R⁵ optionally form, together with the adjacent nitrogen atom, an optionally substituted nitrogen-containing heterocycle,
or a salt thereof.

2. The compound of claim 1, wherein X is —OH.

3. The compound of claim 1, wherein R¹ is a C₃₋₆ alkyl group.

4. The compound of claim 1, wherein R³ is a C₁₋₆ alkyl group.

5. The compound of claim 1, which is
[5-(aminomethyl)-2-ethyl-6-isobutyl-4-(4-methylphenyl)pyridin-3-yl]acetic acid;
[5-(aminomethyl)-2,6-diisobutyl-4-(4-methylphenyl)pyridin-3-yl]acetic acid;
[5-(aminomethyl)-2-ethyl-4-(4-methylphenyl)-6-neopentylpyridin-3-yl]acetic acid; or
1-{[5-(aminomethyl)-2-ethyl-4-(4-methylphenyl)-6-neopentylpyridin-3-yl]acetyl}-L-prolinamide;
or a salt thereof.

6. A prodrug of a compound of claim 1.

7. A pharmaceutical agent comprising a compound of claim 1 or a prodrug thereof.

8. The pharmaceutical agent of claim 7, which is an agent for the prophylaxis or treatment of diabetes, diabetic complications, impaired glucose tolerance or obesity.

9. A peptidase inhibitor comprising a compound of claim 1 or a prodrug thereof.

10. The inhibitor of claim 9, wherein the peptidase is dipeptidyl peptidase-IV.

11. Use of a compound of claim 1 or a prodrug thereof for the production of an agent for the prophylaxis or treatment of diabetes, diabetic complications, impaired glucose tolerance or obesity.

12. Use of a compound of claim 1 or a prodrug thereof for the production of a peptidase inhibitor.

13. A method of preventing or treating diabetes, diabetic complications, impaired glucose tolerance or obesity in a mammal, which comprises administering a compound of claim 1 or a prodrug thereof to said mammal.

14. A method of inhibiting peptidase in a mammal, which comprises administering a compound of claim 1 or a prodrug thereof to said mammal.

15. A method of producing a compound represented by the formula (I-a): 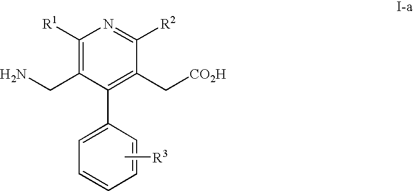
wherein
R¹ is a C₁₋₆ alkyl group optionally substituted by a C₃₋₁₀ cycloalkyl group,
R² is a C₂₋₆ alkyl group, and
R³ is a hydrogen atom, a C₁₋₆ alkyl group or a halogen atom, or a salt thereof, which comprises subjecting a compound represented by the formula (I): 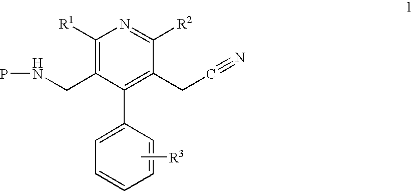
wherein
P is a hydrogen atom or an amino-protecting group, and
R¹, R² and R³ are each as defined above, or a salt thereof, to hydrolysis and deprotection.

16. [5-(aminomethyl)-2-ethyl-6-isobutyl-4-(4-methylphenyl)pyridin-3-yl]acetic acid or a salt thereof.

17. [5-(aminomethyl)-2,6-diisobutyl-4-(4-methylphenyl)pyridin-3-yl]acetic acid or a salt thereof.

18. [5-(aminomethyl)-2-ethyl-4-(4-methylphenyl)-6-neopentylpyridin-3-yl]acetic acid or a salt thereof.

19. 1-{[5-(aminomethyl)-2-ethyl-4-(4-methylphenyl)-6-neopentylpyridin-3-yl]acetyl}-L-prolinamide or a salt thereof.
